(19) United States
(12) Patent Application Publication
(10) Pub. No.: US 2026/0168004 A1
(43) Pub. Date: Jun. 18, 2026

(54) SENSOR STABILITY

(71) Applicant: Glucovation, Inc., Carlsbad, CA (US)

(72) Inventor: Jeffrey Suri, Fallbrook, CA (US)

(73) Assignee: Glucovation, Inc., Carlsbad, CA (US)

(21) Appl. No.: 19/125,434

(22) PCT Filed: Nov. 1, 2023
(86) PCT No.: PCT/US2023/078317

§ 371 Date: Apr. 29, 2025

Related U.S. Application Data
(60) Provisional application No. 63/421,738, filed on Nov. 2, 2022.

(51) Int. Cl. C12Q 1/00; C12Q 1/26; C12Q 1/54

(52) U.S. Cl. CPC C12Q 1/006; C12Q 1/26; C12Q 1/54; C12Y 101/03004; C12Y 113/12004

(57) ABSTRACT

Sensor stability is crucial to successful, long term in situ analyte measurement. Systems, methods and devices herein exhibit increased long term measurement accuracy of circulating analytes, such as glucose, lactose, or others, which makes the technology herein particularly suitable for real time, long term in situ analyte measurement. Systems, methods and devices herein variously comprise crosslinked sensor layers, sensor sites that are internal to rather than at an edge of an insulating layer and other structural improvements, as well as sensors exhibiting the improved long-term stability in analyte measurement resulting therefrom or otherwise facilitating long term use.

### CROSS-REFERENCE TO RELATED APPLICATIONS

This application claims priority to U.S. Provisional Application Ser. No. 63/421,738, filed Nov. 2, 2022, which is hereby incorporated by reference in its entirety.

### BACKGROUND

Accurate ongoing monitoring of analytes, particularly physiologically relevant analytes present in a human subject, is of particular importance to human health. Current approaches require an iterative process of sample removal and assaying which is uncomfortable and which, depending upon timing at which samples are recovered, may fail to capture analytes at time points where their concentrations are highest or lowest, time points which are often of most physiological relevance.

Recent improvements to ongoing monitoring allow a device to be affixed to an individual for an extended period of time, allowing for continuous monitoring. However, probes in these systems are often subject to declines in accuracy over time, often due to loss of bonding of probe constituents and delamination of membranes arising from prolonged hydration and associated swelling.

A major issue for products involving sensor technology is that sensor performance is inconsistent over time. That is, a given analyte amount may be seen to vary over time as a result of degradation of the sensor rather than variation in the actual amount of the analyte. This phenomenon may limit sensor efficacy as well as the time over which an individual sensor may be deployed, for example deployed in situ in an individual.

### SUMMARY

Disclosed herein are systems, methods and compositions related to analyte detection, such as analyte detection in situ within a subject. In many cases an analyte is detected in a liquid solution, such as a body fluid either derived from an individual or in situ within an individual, though detection of analytes in media partially or largely comprising artificial components is also contemplated. Sensors and methods of use of sensors disclosed herein exhibit remarkable stability, both in the capacity to detect a signal over time and in the signal drift which is observed during ongoing detection events. This makes the systems, methods and compositions herein particularly suitable for ongoing internal monitoring of an analyte in an individual, such as circulating lactose, or glucose in the context of diabetes treatment or amelioration. The technology is general, however, and is applicable to a broad range of analytes or conditions for amelioration, treatment or observation.

The disclosure herein is further understood in the context of a number of embodiments.

Disclosed herein are sensors, such as sensors comprising one or more of a solid base; an insulating layer affixed to the solid base, wherein the insulating layer comprises at least one opening that does not share a border with an exterior edge of the insulating layer, so as to expose a portion of the solid base removed from the exterior edge of the insulating layer; a sensor layer comprising a biocompatible polymer and an analyte detection moiety capable of detecting an analyte; and an exterior layer that completely envelops the sensor layer.

A number of solid base configuration are consistent with the disclosure herein. Solid bases may be a wire or needle, or any shape up to and including a wafer or other locally planar or flat surface. Solid bases are often conductive or comprise a conductive component moiety such as one or more conductive traces in a conductive or nonconductive core, such that they may transmit an electric current. This conductive component may also serve as a structural core of the solid base. Alternately, some solid bases comprise a nonconductive solid core that serves a primarily structural function. Such a solid core may have a conductive coating or conductive traces or veins etched or otherwise added to or introduced into the solid core. Conductive solid bases or conductive constituents of solid bases may comprise any number of conductive materials, such as a conductive metal, for example, gold, platinum, iridium, palladium or steel such as stainless steel, although other metals are also consistent with the disclosure herein, particularly metals that are conductive. Nonmetal conductive bases are similarly consistent with the disclosure herein. Alternately, a non-conductive solid metal or nonmetal base that is modified to support a current, though for example a conductive coating or a wiring configuration, is also consistent with the disclosure here.

Current generation is facilitated in some cases by generation of a bias voltage in the sample. Bias voltage is generated using, for example a pair of electrodes or an electrode and the solid base. Suitable voltage biases in various embodiments herein range from near 0 volts to 1 volt, for example less than 0.3 V, less than 0.6 V, or less than, at least, about or exactly, 0.1, 0.2, 0.3, 0.4, 0.5, 0.6, 0.7, 0.8, 0.9 or 1 V.

Some solid bases comprise a component that is both conductive and reactive, such that when in contact with a signaling component such as hydrogen peroxide (H₂O₂), the solid base component reacts with the signaling component to yield an electric charge that may then be conducted by the solid base. Exemplary reactive and conductive base metals include platinum, rhodium, iridium or a combination of these metals. Furthermore, in some cases a conductive, nonreactive metal is used in combination with a reactive constituent such that the reactive constituent reacts with a signaling molecule such as hydrogen peroxide to yield a charge which is then transmitted by the conductive solid base constituent.

An insulating layer is affixed to the solid base, such that in some cases the insulating layer prevents charge from passing through the layer to the solid base. Alternately or in combination, an insulating layer is impermeable to an analyte, such that it precludes an analyte from passing through the insulating layer. Exemplary insulating membranes are plastic, such as polyimide, or alternately polyurethane, PEEK, or Teflon, among or an alternative having similar chemical or insulating characteristics.

As used herein, a layer is a coating or covering that comprises part of a sensor. Layers are alternately permeable or impermeable to one or more signaling molecules, analytes or charge, and if impermeable may be in some cases ablated or punctured to allow the one or more signaling molecules, analytes or charge to traverse the layer specifically at the site of the ablation or puncture. A permeable layer, such as a layer that permits the one or more signaling molecules, analytes or charge to traverse the layer in the absence of a site of ablation or puncture, is in some cases referred to as a membrane. Alternately, in some cases the terms are used interchangeably, such that permeability is a functional distinguishing factor of a component referred to as a layer or membrane.

An insulating layer consistent with the disclosure herein often comprises at least one opening that does not share a border with an exterior edge of the insulating layer. Accordingly, a portion of the solid base, removed from the edge of the insulating layer, is exposed or free of the insulation otherwise effected by the insulating layer, such that charge, analyte or both charge and analyte are accessible through at least one opening, such as a hole in the insulating layer.

Such a hole or opening may be present in an insulating layer prior to contacting to a solid base, such as in cases where the insulating layer is ‘wrapped around’ or ‘ironed on’ or otherwise affixed to a solid base. Alternately, in some cases an unperforated insulating layer precursor is contacted to the solid base, and contacted to an ablation agent to perforate the insulating layer. In particular, when an insulating layer is applied by dipping a solid base into a liquid precursor of an insulating layer, or when a liquid or aerosol precursor of an insulating layer, or other application of a liquid precursor of the insulating layer, is applied to a solid base, then perforation or ablation of the insulating layer is often employed.

A number of ablation agents are consistent with the disclosure herein, such as chemical agents, heat or cold, laser or other electromagnetic energy, or a physical ablation agent that may pierce or abrade a hole into the insulating layer. Other ablation agents and other approaches to perforating the insulating layer are contemplated and consistent with the disclosure herein.

In the case of a wire, a polymer insulating layer may be prepared by laser ablation of, for example, the impermeable polymer polyimide in a discrete pattern that allows for the exposure of the desired surface area of a reactive conductive solid base constituent such as platinum.

For a flat substrate, a photoresist is used along with photolithography to create micropatterns within the polyimide substrate, for example using conventional methods in the art.

Alternately, in some cases a solid base is configured such that a conductive or conductive and reactive component accesses one or more signaling molecules, analytes or charge only at a limited portion of the solid base. In these cases the remainder of the solid base is preferably nonconductive, and the conductive or conductive and reactive component is limited in its access to one or more signaling molecules, analytes or charge by the remaining nonconductive sold base constituent. In these cases the solid base nonconductive constituent comprises the insulating layer in addition to providing some structural rigidity as a solid base constituent.

An insulating membrane may not completely envelop the solid base, such that a current may be transmitted distally from the hole. Nonetheless, an insulating layer coating a wire solid base may cross-sectionally surround the wire so as to provide hoop-rigidity to the structure, even in the vicinity of the hole or opening in the insulating membrane.

Insulating membranes may be nonreactive or alternately be functionalized so as to present a reactive moiety, such as a hydroxy moiety or a carboxy moiety, or other moiety that facilitates covalent binding of an insulating layer to a biosensing layer and in some cases indirectly to an outer layer.

Surface modifications of polymer surfaces to effect surface functionalization have been widely performed using plasma, corona discharge, ultraviolet light, ion beams or bombardment, γ-radiation, and chemical solutions to modify macroscopic surface properties, such as wettability, friction, adhesion, biocompatibility, and molecular recognition. In most cases, the formation of oxygen-containing functional groups, such as —OH and —COOH, occurs on the polymer surfaces.

External to the insulating layer, either as a layer on a locally planar surface or radially from a wire core, for example, is a sensing layer.

A number of sensing layers are consistent with the disclosure herein. In particular, a sensing layer that supports or is compatible with enzymatic activity, and comprises immobilized enzymes is consistent with the disclosure herein. Some sensing layer polymers comprise a hydrogel, such as a negatively charged hydrogel, for example polyacrylate or polyurethane, or other suitable immobilization matrices consistent with the disclosure herein. A sensor layer generally is often permeable to the analyte or porous, so as to allow an analyte to access the hole or opening in the insulating membrane, even when the hole or opening in the insulating membrane is covered by the sensing layer generally.

The sensing layer is often deployed so as not to completely envelop the insulating layer. Consistent with this deployment, the sensor layer is often deployed so as to contact the solid base or to allow analytes to contact the solid base only through the internal hole or opening in the insulating layer.

An analyte detection moiety is often embedded in the sensing layer. Alternately, a detection moiety is tethered to the polymer of the sensing layer, or of another constituent of the sensing layer. A number of analyte detection moieties are consistent with the disclosure herein, and an analyte detection moiety is selected to correlate with a target analyte. Often, an analyte detection moiety is a protein, for example an enzyme or an antibody. In the case where the analyte is glucose, a preferred analyte detection moiety is a glucose oxidase enzyme, although other enzymes that detect or catalyze a transformation of glucose into a moiety suitable for generation of a current in the solid base or otherwise are detectable, for example at the solid base, are also consistent with the disclosure herein. An example, among others, of such an alternate enzyme performing this function is glucose dehydrogenase.

In the case where the analyte is lactate, a preferred analyte detection moiety is a lactate oxidase enzyme, although other enzymes that detect or catalyze a transformation of lactate into a moiety suitable for generation of a current in the solid base or otherwise are detectable, for example at the solid base, are also consistent with the disclosure herein.

A suitable analyte detection moiety reacts with the analyte or catalyzes the generation from the analyte or triggered by the analyte to form a detection product. An exemplary detection product yields a current when contacted to the solid base. In some cases the detection product, or the current, or both the detection product and the current, are in proportion to the amount of the analyte present, so as to allow measurement of the current to indicate the amount of the analyte present, through some or all of a dynamic range. An exemplary detection product is hydrogen peroxide, which is generated by the enzyme glucose oxidase reacting with glucose. Often, the insulating layer is impermeable to the detection product. In contrast, often the sensing layer, or an external biocompatible membrane, or both the sensing layer and an external biocompatible membrane or layer is permeable to the detection product. In some cases the exterior layer is selectively impermeable or impermeable to other products or analytes, such as products that may interfere with measurement of the desired analyte or detection of the signaling molecule.

Sensing layers are in some cases nonreactive or inert. Alternately, in some exemplary embodiments the sensing layer is functionalized with a reactive moiety, such as a moiety compatible with cross-linking to a reactive moiety on the insulating layer. Exemplary reactive moieties include carboxy or carboxylate moieties, or hydroxy or hydroxyl moieties, although others are also consistent with the disclosure herein. Consistent with these reactive moieties, in some cases the sensor layer is covalently bound to the insulating membrane.

External to the sensing layer, either as a layer on a locally planar surface or radially from a wire core, for example, is an exterior membrane or layer. An exterior layer is often permeable to the analyte or porous so as to allow the analyte to pass through to the sensing layer, facilitating generation of a detection product.

Exterior membranes are in some cases nonreactive or inert. Alternately, in some exemplary embodiments the sensing layer is functionalized with a reactive moiety, such as a moiety compatible with cross-linking to a reactive moiety on the exterior membrane or layer. Exemplary reactive moieties include vinyl moieties, aziridine moieties, or epoxy moieties, although others are also consistent with the disclosure herein. Consistent with these reactive moieties, in some cases the sensing layer is covalently bound to the exterior membrane.

Sensors as disclosed herein often comprise at least one hole in the insulation layer, in some cases at least two, at least three or at least four holes, such as at least 1, 2, 3, 4, 5, 6, 7, 8, 9, 10 or more than 10, or no more than 1, 2, 3, 4, 5, 6, 7, 8, 9, 10, or exactly 1, 2, 3, 4, 5, 6, 7, 8, 9, or 10.

Sensors as disclosed herein exhibit substantial sensor stability, such that in ongoing measurement conditions, some sensor results do not exhibit substantial drift. Ongoing measurement is performed in vitro or, in exemplary cases, in situ in an individual. In some cases signal drift is no more than 1% per hour. Some sensors disclosed herein exhibit signal drift of no more than 1%, 2%, 3%, 4%, 5%, or 6% signal drift. Alternately, some sensors exhibit signal drift of no more than 1%, 0.9%, 0.8%, 0.7%, 0.6%, 0.5%, 0.4%, 0.3%, 0.2%, or 0.1% per hour. This low signal drift is observed even after 1, 2, 3, 4 or more than four days of measuring, in particular after 1, 2, 3, 4 or more than four days of measuring an analyte in situ in an individual.

Also disclosed herein are methods of making sensors, such as the sensors described above or elsewhere herein. Some such methods comprise one or more of the following elements: contacting a solid base to an insulating agent to form an insulating layer on the solid base; forming at least one hole in the insulating layer such that the solid base is exposed at the hole; contacting the insulating layer to a detection precursor comprising an immobilization polymer precursor and an analyte detection moiety capable of detecting an analyte; polymerizing the polymer precursor to form a sensing layer that spans at least part of the insulating layer and the at least one hole in the insulating layer; crosslinking at least a portion of the sensor layer to at least a portion of the insulating layer; contacting the sensor layer to an exterior layer agent to form an exterior layer, and crosslinking at least a portion of the sensing layer to at least a portion of the exterior biocompatible layer. In some cases the crosslinking is performed in succession. Alternately, in some cases all layers are deposited and then all of the crosslinking occurs. For example, crosslinking may be effected by depositing all the layers, subjecting the deposited layers, membranes, or layers and membranes to oxidation so as to generate functional moieties, and then crosslinking compatible functional moieties to one another. A result of these approaches is that a sensor comprises a chemically fused system, such that two or more layers are covalently attached to one another and to the insulating layer, which often conveys stability and accuracy over time to the sensor system.

Contacting the solid base to the insulating agent variously comprises spraying, dispensing, or spin-coating the insulating agent onto a flat surface of the solid base or dipping the solid base into a liquid composition of the insulating agent, and in either case then polymerizing the insulating agent or allowing the insulating agent to dry on the solid base. The solid base is, as above, in some cases cylindrical, such as a wire or needle, or locally planar. The solid base is or comprises an electrical conductor. In some cases, the solid base comprises a core and a clad. The core provides rigidity and robustness while the clad provides electrical conductivity, and may also provide a reactive surface at which the electrochemical reaction occurs. Exemplary clad metals are platinum, iridium, rhodium, a combination of metal and rhodium, or other metal or nonmetal reactive conductors. Exemplary core materials are platinum, rhodium, a combination of metal and rhodium, iridium, a combination of iridium and platinum, tantalum, nitinol, stainless steel or other metal or nonmetal or nonconductors that may confer structural rigidity to the sensor. Alternately, some bases comprise a conductive moiety in addition to or alternative to a conductive or conductive and reactive coating.

The deposited insulating layer is not an electrical conductor, and is preferably impermeable to the analyte and to a detection moiety. Similarly, the insulating layer is often impermeable to a solvent. The deposited insulating layer often comprises a polymer or is polymerized from monomer units to form a polymer. Exemplary insulating layer comprise a plastic, such as polyimide or other constituent discussed herein. Often, the insulating layer is a polymerization product of the insulating agent or a solidified product of the insulating agent.

At least one hole is formed internal to an edge of the insulating layer. The at least one hole is formed in some cases via etching, such as chemical etching, laser etching, heat etching, or mechanical etching, or a combination of these approaches. Alternately or in combination, an insulation layer may be deposited by pouring an insulation agent onto a solid base and allowing it to cure into shape around the solid base. In particular, in some cases the solid base is placed into a mold and then the insulating layer precursor is poured into the mold.

Alternately, particularly in the case of a locally planar surface, a flat polyimide base material may be deposited and then platinum or other electrode material, or platinum and other electrode material is electrodeposited into a surface such as a well to make an electrode functional polyimide substrate. An example of an electrode functional polyimide substrate is shown in FIG. 1A and FIG. 1B. Forming the whole often comprises ablation, such as laser ablation, of the insulating layer.

Independent of how the at least one hole is formed, the hole made through any of the methods above provides access to the solid base by a solvent or carrier, such as a solvent or carrier in which an analyte is suspended or dissolved.

A number of insulating layer delivery methods are consistent with the disclosure herein. For example, a solution of insulation layer precursor such as such as polyamic acid, a polyimide precursor, is dip-coated onto the substrate via a reel-to-reel system and then heat is applied to remove solvent and solidify the polymer on the surface of the base metal as a thin coating.

The insulating layer formed thereby optionally comprises a functionalizing moiety. The functionalizing moiety is in some cases attached to the insulating agent prior to polymerization or solidification. Alternatively, the agent may be added through a chemical reaction, such as oxidation, performed on the polymerized or solidified insulating layer. This modification variously occurs prior to or subsequent to contacting the insulating layer to the sensing layer or the detection precursor.

Functionalized moieties facilitate the bonding such as covalent bonding of the insulating layer to the sensing layer. Exemplary functionalizations include hydroxy moieties and carboxy moieties. Crosslinking variously occurs concurrently with or subsequent to contacting the insulating layer to the sensing layer. An effect of this crosslinking is to stabilize the sensing layer bound to or around the insulating layer, thereby enhancing the stability of the sensor and accuracy over time of the senor.

A crosslinking configuration is shown in FIG. 2.

The sensor is in some cases formed by contacting a sensing layer precursor to the solid base coated by the insulating layer.

Contacting the solid base and the insulating agent to the sensing layer variously comprises spraying the sensing layer precursor agent onto a flat surface of the insulated layer on the solid base or dipping the insulated layer and solid base into a liquid composition of the sensing layer precursor agent, and in either case then polymerizing the sensing layer precursor agent or allowing the sensing layer precursor agent to dry on the solid base. Alternately, in some cases a prefabricated sensing layer is wrapped or ironed on to the insolated layer of the solid base.

In exemplary embodiments, the sensing layer is deposited so that sensing signal does not reach the solid base other than through a hole in the insulating layer. For example, the sensing layer is deposited in some cases so as not to engulf the insulating layer, or otherwise so as to not access the conductive portion of the solid base other than through a hole internal to the insulating layer. Other configurations that restrict communication between the sensor layer and the solid base are also contemplated and consistent with the disclosure herein.

The sensing layer is in some cases cross-linked or bound to the insulating layer through functionalizing groups. The functionalizing group is in some cases present in the sensing layer precursor. Alternately, the functionalizing group is added to the sensing layer pursuant to polymerization or subsequent to polymerization. Exemplary functionalizing groups comprise hydroxy moieties, carboxy moieties, aziridine moieties, vinyl moieties, epoxy moieties, though any functionalizing group that may bind to a complementary functionalizing group of the insulating membrane is consistent with the disclosure herein, particularly a functionalizing agent that may be bound to the insulating layer without abolishing bioactivity, such as sensing moiety activity, in the sensing layer. Exemplary sensing layer precursors comprise one or more of hydroxy moieties, carboxy moieties, aziridine moieties, vinyl moieties, epoxy moieties. External biocompatible layers optionally also comprise a hydrophilic component, so as to maintain semi-permeability of the membrane, particularly to a solvent, a carrier, or an analyte or detection agent, for example an aqueous solution comprising an analyte such as glucose, lactate, or both glucose and lactate, such as an interstitial fluid.

The sensing layer comprises a sensing moiety. Attaching the sensing moiety to the sensing layer variously comprises delivering the sensing moiety concurrently with the sensing layer polymer precursor or delivering the sensing moiety subsequent to delivery or subsequent to polymerization or solidification of the sensing layer precursor. Exemplary sensing moieties are proteins, such as enzymes or antibodies, or synthetic receptors such as boronic acids, that retain some activity pursuant to polymerization, functionalization or cross-linking to the insulating layer or the external membrane or both.

As an example of how crosslinking may be effected, the following is provided. It is understood that alternatives are known in the art and consistent with the disclosure herein. The reaction of polyfunctional aziridine cross-linkers are acid catalyzed. They are typically used to react with the carboxylic acid groups on acrylic adhesives. To cross-link, an active hydrogen may be available to open the aziridine ring. The exemplary reaction mechanism is shown below:

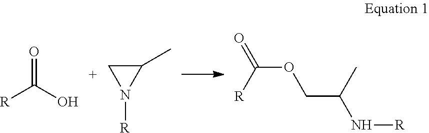

The active hydrogen on the carboxylic acid group on the acrylic or polyurethane resin reacts with the nitrogen of the polyfunctional aziridine, which opens the ring to cross-link the resin. Since polyfunctional aziridines are tri-functional and the acrylic emulsion of polyurethane dispersion or polyacrylic acid can be multi-functional, a cross-link density or network is formed through this reaction mechanism. The polyfunctional aziridine and carboxylic acid reaction will occur at ambient condition as the coating, ink, or adhesive dry. Adding heat in the drying process will increase the rate of reaction. As the system dries, the pH drops, which accelerates the reaction of the active hydrogen of the carboxylic acid with the polyfunctional aziridine.

This reaction mechanism may happen internal to the sensing layer and may also occur on the surface of the oxidized polyimide surface; in both cases a carboxy group is being crosslinked with the polyfunctional aziridine.

Alternately, a more elaborate cross-linking scheme comprises a tie layer in between the polyimide and the sensing layer. In this system, more extensive cross-linking moieties are used, constituting an intervening ‘tie layer’ comprising silane attaching to hydroxy groups having carboxy groups at the other end, and this then reacts along with carboxy groups in the sensing layer. Exemplary tie layer configurations are shown in FIG. 3 and in Table 1, below.

As mentioned above, this reaction mechanism is provided as an exemplary embodiment. Various other crosslinking chemistry technologies are also known and compatible with some or all of the disclosure herein.

Various permutations of tie layers in combination with the core structures disclosed herein are presented below. A general feature of these intervening layers is that they often add stability, such as via covalent attachment or provide a source of additional chemistry to effect crosslinking or otherwise increase stability of the sensor.

TABLE 1

| Structure 1 | Structure 2 | Structure 3 | Structure 4 |
|---|---|---|---|
| | | | External Layer |
| | External Layer | External Layer | Primer Layer |
| External Layer | Primer Layer | Sensing Layer | Sensing Layer |
| Sensing Layer | Sensing Layer | Tie Layer | Tie Layer |
| Substrate | Substrate | Substrate | Substrate |

Alternately, some layers may be relied upon to provide additional functionality.

The sensor is in some cases formed by contacting an exterior membrane precursor to the solid base coated by the insulating layer and the sensing layer.

Contacting the assembly of the solid base, the insulating agent and the sensing layer to the exterior membrane precursor variously comprises spraying an exterior membrane precursor onto a flat surface of the sensor precursor or dipping the sensor precursor into a liquid composition of the exterior membrane precursor, and in either case then polymerizing the exterior membrane precursor or allowing the exterior membrane precursor to dry on the sensor precursor. Alternately, in some cases a prefabricated exterior membrane is wrapped or ironed on to the sensor precursor, and additional application approaches are contemplated and consistent with the disclosure herein.

The exterior biocompatible membrane layer is in some cases functionalized, so as to bind to the sensing layer. Functionalization is effected by functionalizing the exterior layer precursor. Alternately, the exterior layer is functionalized pursuant to polymerization or solidification, or subsequent to polymerization or solidification. Exemplary functionalizing moieties include vinyl moieties and hydride moieties, though any moiety that is able to cross-link or bind to sensing layer functional moieties are consistent with the disclosure herein, particularly when the crosslinking is effected without abolishing biosensing activity of the sensing layer.

As an additional example, the exterior membrane may be polymerized via a hydrosilylation reaction where a vinyl silicone prepolymer is reacted with a hydride silicone prepolymer. A vinyl functional hydrophile is included as part of the reaction mixture in order to render the final polymer membrane semi-permeable to the analyte of interest and simultaneously covalently attach the exterior membrane to the sensing layer.

The layering and cross-linking convey a degree of durability to the sensors, such that they are stable and accurate for long-term use, such as use in vivo.

Disclosed herein, accordingly, are sensors that exhibit durability or stability so as to facilitate long term use, for example, use deployed in vivo in an individual. Such sensors are resilient to degradation that may arise from biofouling, layer separation or other challenges to structural integrity or performance.

Sensors in some cases exhibit a signal drift of no more than 1% of signal/hour. Some such sensors exhibit a signal drift of no more than 0.9%, 0.8%, 0.7%, 0.6%, 0.5%, 0.4%, 0.3%, 0.2%, 0.1% or less than 0.1% per hour. This low signal drift is observed, for example, in sensing glucose or lactate or other analyte at a concentration of about 400 mg/dL, or alternately about 300 mg/dL, about 200 mg/dL, about 100 mg/dL, or less than 100 mg/dL or at least 40 mg/dL. In some cases the sensor comprises crosslinked layers, such as crosslinked layers disclosed above. Similarly, in some cases the sensor is generated through a process comprising crosslinking of two or more layers to one another.

Signal to be detected is in some cases accessible to a conducting moiety only though a gap or hole in an insulating layer, such as a gap or hole that maintains its local structural integrity due to one or more than one layers superimposed on top of the hole and in some cases cross-linked to the insulating layer or to a layer external to the insulating layer, so as to provide structural integrity to a sensor at the measurement site.

Similarly, signal to be detected is in some cases generated in a signal layer that maintains its local structural integrity due to one or more than one layers superimposed on top of or beneath it and in some cases cross-linked to the signal layer, so as to provide structural integrity to a sensor at the sensing site.

Similarly disclosed herein are methods for extending sensor performance comprising making or using the sensors as disclosed herein.

Similarly disclosed herein are methods for detecting analytes in a sample external to or internal to an individual. Some such methods comprise one or more of the steps of creating a bias voltage in the sample, and measuring a current passing to a conductive base through at least one opening in an insulating layer, wherein the at least one opening does not share a border with an exterior edge of the insulating layer. Suitable samples include body fluids such as circulating blood, and may be performed in a fluid internal to an individual. Bias voltages of from above 0 to 1 volt are contemplated herein, such as no more than 1, 0.9, 0.8, 0.7, 0.6, 0.5, 0.4, 0.3, 0.2, 0.1, or less than 0.1, as well as at least or no more than 0.001, 0.005, 0.01, 0.05, 0.1, or greater than 01. Other bias voltages are contemplated and consistent with various embodiments of the disclosure herein. Bias voltage is generated using, for example a pair of electrodes or an electrode and the solid base.

Current arises at the opening in the insulating layer through contacting of a secondary signaling molecule such as an oxidation product. Current formation is facilitated by the generation of a bias voltage in the sample, in particular where the solid base is in ‘pole’ of the bias voltage and the other is in the sample solution. A number of oxidation products are consistent with the disclosure herein, such as hydrogen peroxide. Signaling molecules are variously generated through reactions on the analyte or analytes, such as enzymatic reactions. Reactions often comprise the action of an oxidase, such as a glucose oxidase or lactate oxidase. More exhaustive lists of potential enzymatic reaction constituents are disclosed elsewhere herein, and additional enzymatic participants are also contemplated in light of the art.

Such reactions would generate a signaling molecule population, and often a charge, in proportion to the amount of analyte within or in proximity to the opening or openings in the insulating layer. Thus, the current is often proportionate to the concentration or amount of analyte molecules in the vicinity of the opening or openings in the insulating layer.

Often, diffusion of the analyte or the signaling molecule in the vicinity of an opening in the insulating layer is limited, for example so as to facilitate accurate quantification. Limiting diffusion variously comprises covering the opening using an external layer, such as an external layer permeable or semipermeable to the analyte.

The current is then conducted by the base or by a channel or channels within the base to a sensor. The insulating layer precludes current from passing to the conductive base other than through the at least one opening, such that current is proportional to the amount of analyte or signaling molecule at the one or more openings. Thus, the methods allow for current to be measured as an indicator of presence and amount of analyte.

Suitable analytes include, for example, glucose or lactate, though a broad range of analytes may be detected using the methods herein.

Methods herein allow ongoing, reliable detection of an analyte, such as in situ in an individual, for extended periods of time. Detections are variously performed continuously or intermittently on a sample in an individual for at least 6, 16, 18 or 24 hours, or 2, 3, 4 or more than 4 days. Such prolonged or ongoing practice of the detection methods exhibit a signal drift of no more than 1% of signal per hour, or levels of signal drift specified elsewhere herein, after at least 6, 16, 18 or 24 hours, or 2, 3, 4 or more than 4 days. Low levels of signal drift with ongoing or prolonged signal detection are achieved at least in part due to selection of sites for measuring current passage to a conductive base through at least one opening in an insulating layer, wherein the at least one opening does not share a border with an exterior edge of the insulating layer. By selecting at least one opening in an insulating layer that does not share a border with an exterior edge of the insulating layer, one may perform ongoing, reliable detection as the detection site retains its structural integrity over time, particularly in comparison to at least one opening in an insulating layer, wherein the at least one opening shares a border with an exterior edge of the insulating layer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

In particular, US2022/0167886A1, published Jun. 2, 2022, relates to chemistries consistent with the disclosure herein, and is incorporated by reference in its entirety.

### BRIEF DESCRIPTION OF THE DRAWINGS

FIG. 1A. Needle sensor configuration.

FIG. 1B. Locally planar sensor configuration.

FIG. 2. Crosslinking configuration.

FIG. 3. Tie layer crosslinking configuration.

FIG. 4. 1 mm Tip exposure wire schematic.

FIG. 5A. 1 mm Tip exposure wire current measurement

FIG. 5B. 1 mm Tip exposure wire current measurement

FIG. 6. 4× slit hole exposure wire schematic

FIG. 7A. 4× slit hole exposure wire current measurement

FIG. 7B. 4× slit hole exposure wire current measurement

FIG. 7C. 4× slit hole exposure wire current measurement

FIG. 8. 2× slit hole exposure wire

FIG. 9A. 2× slit hole exposure wire current measurement

FIG. 9B. 2× slit hole exposure wire current measurement

FIG. 9C. 2× slit hole exposure wire current measurement

FIG. 10. 1× skived exposure wire

FIG. 11A. 1× skived exposure wire current measurement

FIG. 11B. 1× skived exposure wire current measurement

FIG. 12. A schematic of a glucose sensing system

FIG. 13. A Clark Error Grid for a 4-hole slit probe.

FIG. 14. Data from a sensing system herein configured to detect lactate.

### DETAILED DESCRIPTION

Accurate, ongoing monitoring of a target analyte in situ in an individual or other subject requires a durable, accurate sensor. The sensor must be able to make ongoing measurements and be resistant to the destructive effects of the local environment. To survive in such an environment, a multilayered sensor must be resistant to the delamination effects that otherwise result from swelling and hydration that result from prolonged contact with blood or other subject bodily fluids.

The disclosure herein addresses these challenges, both through an improved mechanical structure and through am improved chemical relationship among sensor constituents. Accordingly, embodiments incorporating mechanical structural improvements, or embodiments incorporating improved chemical relationship among constituents, or embodiments incorporating both mechanical structural improvements and improved chemical relationships among constituents, represent a clear improvement in the art.

Analyte detection sensors operate by converting presence of an analyte into a detectable, quantifiable signal. Often this process of converting comprises chemically catalyzing the creation of a detectable signal molecule from a target analyte at a predictable rate relative to the amount of analyte, such that detection of the detectable signal may indicate amount of the analyte in a sample. Such a signal may generate a current, particularly when a bias voltage is generated in a sample such that signaling molecules such as hydrogen peroxide may yield a current when in contact with a conductive solid base. Many approaches for the use of bias voltage to create an environment in which a current may be generated from, for example, an oxidation product are well known in the art

A number of analytes may be targeted for measurement using the general sensor structures disclosed herein. An analyte that has received particular attention is glucose, notable for its harm effects when not properly regulated by insulin, as is the case with individuals suffering from diabetes. Other analytes, such as lactate or any analyte that may be involved in a reaction so as to generate a current, either directly or through a detectable signal molecule such as hydrogen peroxide (H₂O₂), are consistent with the disclosure herein.

Glucose is readily acted on by the enzyme glucose oxidase, which catalyzes the generation of hydrogen peroxide (H₂O₂) in an equimolar amount to the amount of glucose present. Hydrogen peroxide then acts as detectable signal molecule, which yields a current when in contact with a conductive solid base.

The disclosure is consistent with glucose detection, but is also applicable to a broad number of target analytes from which a quantitative signal can be generated. A quantitative signal may comprise a reactive signaling molecule that is produced upon contacting of the target analyte to a detector such as an enzyme. The reactive signaling molecule may then yield a charge upon contact with a reactive solid base constituent.

A number of examples of enzymes suitable for detection of their indicated target analytes through the release of the oxidatively reactive species hydrogen peroxide are listed below. As a partial list, they include enzymes of the oxidoreductase EC1 class such as glucose oxidase, lactate oxidase, malate oxidase, glucose oxidase, hexose oxidase, cholesterol oxidase, aryl-alcohol oxidase, L-gulonolactone oxidase, galactose oxidase, pyranose oxidase, L-sorbose oxidase, pyridoxine 4-oxidase, alcohol oxidase, catechol oxidase (dimerizing), (S)-2-hydroxy-acid oxidase, ecdysone oxidase, choline oxidase, secondary-alcohol oxidase, 4-hydroxymandelate oxidase, long-chain-alcohol oxidase, glycerol-3-phosphate oxidase, xanthine oxidase, thiamine oxidase, L-galactonolactone oxidase, columbamine oxidase, hydroxyphytanate oxidase, nucleoside oxidase, N-acylhexosamine oxidase, polyvinyl-alcohol oxidase, (S)-stylopine synthase, (S)-cheilanthifoline synthase, berbamunine synthase, salutaridine synthase, (S)-canadine synthase, D-arabinono-1,4-lactone oxidase, vanillyl-alcohol oxidase, nucleoside oxidase (H₂O₂-forming), D-mannitol oxidase, xylitol oxidase, prosolanapyrone-II oxidase, paromamine 6′-oxidase, 6′″-hydroxyneomycin C oxidase, aclacinomycin-N oxidase, 4-hydroxymandelate oxidase, 5-(hydroxymethyl) furfural oxidase, 3-deoxy-α-D-manno-octulosonate 8-oxidase, (R)-mandelonitrile oxidase, aldehyde oxidase, pyruvate oxidase, oxalate oxidase, glyoxylate oxidase, pyruvate oxidase (CoA-acetylating), indole-3-acetaldehyde oxidase, pyridoxal oxidase, aryl-aldehyde oxidase, retinal oxidase, vanillate monooxygenase, abscisic-aldehyde oxidase, (methyl)glyoxal oxidase, dihydroorotate oxidase, lathosterol oxidase, coproporphyrinogen oxidase, protoporphyrinogen oxidase, bilirubin oxidase, acyl-CoA oxidase, dihydrouracil oxidase, tetrahydroberberine oxidase, secologanin synthase, tryptophan a,b-oxidase, pyrroloquinoline-quinone synthase, D-aspartate oxidase, L-amino-acid oxidase, D-amino-acid oxidase, monoamine oxidase, pyridoxal 5′-phosphate synthase, D-glutamate oxidase, ethanolamine oxidase, putrescine oxidase, L-glutamate oxidase, cyclohexylamine oxidase, protein-lysine 6-oxidase, L-lysine oxidase, D-glutamate (D-aspartate) oxidase, L-aspartate oxidase, tryptophan α,β-oxidase, glycine oxidase, L-lysine 6-oxidase, primary-amine oxidase, diamine oxidase, 7-chloro-L-tryptophan oxidase, pseudooxynicotine oxidase, L-arginine oxidase, pre-mycofactocin synthase, sarcosine oxidase, N-methyl-L-amino-acid oxidase, N-6-methyl-lysine oxidase, (S)-6-hydroxynicotine oxidase, (R)-6-hydroxynicotine oxidase, L-pipecolate oxidase, reticuline oxidase, dimethylglycine oxidase, dihydrobenzophenanthridine oxidase, NI-acetylpolyamine oxidase, polyamine oxidase (propane-1,3-diamine-forming), N8-acetylspermidine oxidase (propane-1,3-diamine-forming), spermine oxidase, non-specific polyamine oxidase, L-saccharopine oxidase, 4-methylaminobutanoate oxidase (formaldehyde-forming), N-alkylglycine oxidase, 4-methylaminobutanoate oxidase (methylamine-forming), coenzyme F420H2 oxidase, glyphosate oxidoreductase, nitroalkane oxidase, acetylindoxyl oxidase, factor-independent urate hydroxylase, 3-aci-nitropropanoate oxidase, hydroxylamine oxidase (cytochrome), catechol oxidase, laccase, L-ascorbate oxidase, o-aminophenol oxidase, 3-hydroxyanthranilate oxidase, rifamycin-B oxidase, ubiquinol oxidase (non-electrogenic), grixazone synthase, dihydrophenazinedicarboxylate synthase, superoxide oxidase, reticuline oxidase, isopenicillin-N synthase, columbamine oxidase, reticuline oxidase, aureusidin synthase, tetrahydrocannabinolic acid synthase, cannabidiolic acid synthase, dichlorochromopyrrolate synthase. Generally enzymes from the 7 classes (oxidoreductases, transferases, hydrolases, lyases, isomerases, ligases, and translocases) can be immobilized into the enzyme layer and utilized for detection of target analytes.

Consistent with the broad range of enzymatic detection moieties seen above, a wide range of analytes for detection are compatible with the disclosure herein. These analytes include glucose most prominently, but also lactate and a broad range of monosaccharides, carbohydrates, alcohols, amino acids, enantiomers, and small molecule metabolites as indicated or understood to be substrates of the enzymes listed above, among other examples of analytes for detection. Many enzymes or nonenzymatic detection moieties act through the release of a reactive oxidized product such as hydrogen peroxide. Alternatively, some enzymes such as dehydrogenase enzymes or nonenzymatic detection moieties yield electrons directly, which may lead to current generation in a reactive or even a nonreactive conductive solid base constituent. Examples of these detection moieties include a broad range of dehydrogenase enzymes, as well as nonenzymatic detection moieties. An exemplary nonenzymatic detection moiety is boronic acid, which serves as a glucose receptor and when coupled to a redox system can display a change in its electronic state upon contact with glucose.

Accordingly, hydrogen peroxide, molecules that involve electron transfer, and a number of related molecules that may directly or indirectly give rise to a charge that may be detected upon contacting to a conductive solid core constituent such as a reactive conductive solid core constituent are contemplated and consistent with the disclosure herein. Broader examples of detection moieties are also contemplated, such as polymerase/primed nucleic acid template complexes, which may detect a complementary base by incorporating that base into the primer, yielding a phosphate moiety as a signal molecule.

Generally, any analyte that is amenable to enzymatic or other generation of a detectable signal molecule is suitable for detection using a probe herein. One class of detectable signal molecules readily yield a current when in contact with a conductor solid base. Hydrogen peroxide is an example, as are a number of oxidizing or reducing molecules that may contribute or accept an electron from a conductive medium.

In the embodiments contemplated above the detectable signal is a current, which is generated upon contacting of a detectable signal molecule to a probe constituent that is capable of conducting a current. Current generation is facilitated in some cases by generation of a bias voltage in the sample, such as a bias voltage in the range of about 0.1, 0.2, 0.3, 0.4, 0.5, 0.6, 0.7, 0.8, 0.9, or 1 Volt, or a bias voltage outside of this range. Metallic solid bases are preferred for receiving current from detectable molecules and transmitting that current distally from the detection site to where the current can be quantitatively measured. Platinum and rhodium are preferred solid base embodiments, though any number of conductive metals are consistent with the disclosure herein, as are nonmetal conducting solid base materials. Some additional solid base examples consistent with the disclosure herein include carbon paste, silver paste, and conductive polymers such as polyaniline, polypyrrole and poly(3,4-ethylenedioxythiophene), poly(3,4-ethylenedioxythiophene)-poly(styrenesulfonate), and polythiophene.

A number of nonenzymatic or nonreactive detection systems are also contemplated. It should be understood that nonelectric detectable signals are also contemplated, along with non-oxidatively reactive detectable signal molecules. For example, some detection systems consistent with the disclosure herein comprise binding of an analyte to an antibody rather than to an enzyme.

Antibodies are large Y-shaped proteins produced by plasma cells that are utilized by the immune system to identify and target pathogens such as bacteria and viruses. Their small size, high stability and easy genetic manipulation make recombinant antibody fragments valuable and robust tools for the fabrication of immunosensors. Antibody-based biosensors have revolutionized diagnostics for the detection of a plethora of analytes such as food and environmental contaminants, biological warfare agents, illicit drugs and disease markers. Immobilization of antibodies onto a sensor surface without altering their specificity and immunological activity is one of the most crucial steps in the fabrication of a successful immunosensor. The immobilization step affects the detection limit, sensitivity and overall performance of the immunosensor. Orientation of antibodies on sensor surfaces can be controlled by the interaction between specific reactive groups on the surface and on the antibody.

Antibodies may generate signal moieties by, for example being coupled to a reagent that, in an antibody/analyte-binding specific manner, generates a charge or a detectable species such as hydrogen peroxide that reacts with a reactive solid base constituent to generate a current in the solid base. One example of this is the enzyme-linked immunosorbent assay (ELISA), an immunological assay commonly used to measure antibodies, antigens, proteins and glycoproteins in biological samples.

As antibodies may be generated to target a broad range of antigens, use of antibodies in detection further expands the range of analytes that may be assayed for using the systems and methods herein.

Sensor Stability A feature common to a number of these detection mechanism is that they are chemically harsh. That is, they subject sensors to stresses such as oxidative stresses that may serve to degrade the sensors over time, impacting their accuracy. Both hydrogen peroxide and molecules that involve electron transfer (such as redox reactions) are reactive and may act not only on the solid base but on other constituents of a sensor system, to the detriment of long term sensor stability and accuracy.

In addition, the environment from which target analytes are drawn, such as blood, may itself serve to degrade sensors over time. This is particularly true when detector layers absorb solvent or carrier liquid, as this absorption may result in expansion or delamination (‘wrinkling’) of various sensor layers. Any of these phenomena may impact solid base reaction zone size, local volume, reagent accessibility, or otherwise challenge or impact the long-term accuracy of a sensor.

Probe accuracy relies upon a known, defined area upon which a detectable signal molecule may act to generate a current or other conductible signal. Variation in this area, for example by separation of insulating layers that form a border of this area, may result in signal drift over time, as the reactive area changes. Without being bound by theory, separation may result from, for example, degradation by oxidative stress caused by a signal detection molecule, or delamination of one or more layers due to absorption of an analyte solvent or carrier liquid.

Exposing an end of a wire probe or edge of a flat detection surface is vulnerable to degradation because there is little structural integrity to prevent the insulation membrane at the edge of the detection area from fraying or delaminating. Structural tension is at best applied only parallel to the insulation border edge. As the insulation layer expands through absorption of water or degrades through contact to oxidative molecules or current, there may be a decrease in the structural tension holding the insulating membrane in place at the sensor area border. This may result in a change in the sensor area, impacting sensor accuracy over time.

Disclosed herein is technology related to maintaining sensor integrity in the face of long-term use. Two aspects of this technology, operating alone or in combination, comprise mechanical structural stabilization and chemical structural stabilization.

Probe configurations disclosed herein in some cases facilitate probe structure and prolonged maintenance of probe measurement accuracy by maintaining structural integrity of the detection area borders. This is accomplished by stabilizing an insulating layer in the vicinity of a detection area. As a result, there is reduced variation in detection area size over time, which may lead to more accurate measurements.

Mechanical Structural Stabilization. Mechanical structural stabilization is effected by increasing the number of directions from which tension on an insulating membrane may hold the borders of a detection area in place. This is accomplished by generation of a hole in an interior section of an insulation membrane, such that the edges of the detection area are distal to the exterior edges of the insulation membrane. The borders of this hole then define the edges of the detection area. Because the detection area is bound on all sides by a continuous insulation membrane, the membrane provides tension pulling against or counteracting any force acting to pull the insulation membrane away from the solid base. Without being bound by theory, it is likely that this serves to preserve the detection area by preventing the insulation membrane from peeling off of the solid base.

Generating a sensor having a hole whose edge is internal to the insulating membrane and not continuous with an external edge of the insulating membrane is accomplished through a number of approaches.

In some probe configurations herein, a pre-perforated insulating membrane is wrapped onto a cylindrical solid base, such as a wire, or adhered or ‘ironed on’ to a locally planar surface. The perforation or perforations then define the sensor area on the solid base.

Alternately, a hole or holes are generated by ablation of a generally applied insulation membrane. The membrane may arise from a monomeric precursor, such as a monomer in a solution into which a wire solid base is dipped, or which is sprayed, painted or otherwise deposited onto a locally planar surface. The insulation membrane is allowed to polymerize or solidify, and then is locally ablated to form one or more internal holes.

Ablation is effected through a number of approaches known in the art. Ablation may be effected through application of heat, pressure, force, electromagnetic energy, laser etching, or chemical etching such as etching using an acid or other reactive agent. Ablation is characterized by the creation of a hole in an insulating layer of known size or at least size that can be modulated. The hole must be sufficiently deep to allow the analyte or the signal indicative of the analyte to pass through. Examples of ablation consistent with the disclosure herein include, for example, laser ablating, photochemical machining, sputtered deposition, electrochemical plating and deposition, and photolithography.

Chemical Structural Stabilization. Chemical structural stabilization is effected by chemically binding two or more layers of a sensing instrument to one another, such that structural integrity of one layer is conveyed to other layers, in particular the insulating layer.

Sensors disclosed herein variously comprise one or more of a solid base, an insulating membrane, a sensing layer and an exterior membrane. By crosslinking one or more layers, one may increase the structural integrity of the sensor as a whole and of the sensing area in particular.

Crosslinking is effected by reacting functional groups on adjacent layers so that the layers or membranes become chemically bound at the functionalization groups.

Insulating layers, often comprise an impermeable polymer, such as a plastic, so as to limit access to the conductive solid base. Exemplary impermeable layers include polyurethane, PEEK, Teflon, or, often, polyimide. This membrane or layer is optionally functionalized using hydroxy or carboxy moieties. These moieties may be added to monomeric constituents or may be added concurrent with or subsequent to polymerization.

Adjacent sensor layers often comprise a biocompatible hydrogel, such as polyacrylate or polyurethane. This layer is optionally functionalized using carboxy moieties. These moieties may be added to monomeric constituents or may be added concurrent with or subsequent to polymerization. More generally, any functional group that may covalently or noncovalently bind to a functional group on the insulating layer or exterior membrane, and which is not inconsistent with detection moiety activity, is consistent with the disclosure herein.

Adjacent to the sensor layer is often an exterior membrane. Exterior membranes may be functionalized so as to covalently or noncovalently bind to a functional group on the sensing layer. Exemplary functionalizing moieties include vinyl moieties and hydride moieties. More generally, any functional group that may covalently or noncovalently bind to a functional group on the sensing layer, and which is not inconsistent with detection moiety activity, is consistent with the disclosure herein.

Without being bound by theory, cross-binding among layers of a sensor serve to stabilize the sensor, such that readings remain consistent over time, as is needed for use in a device that is to be performing long-term measurements on a subject.

Turning to the Figures, one sees the following.

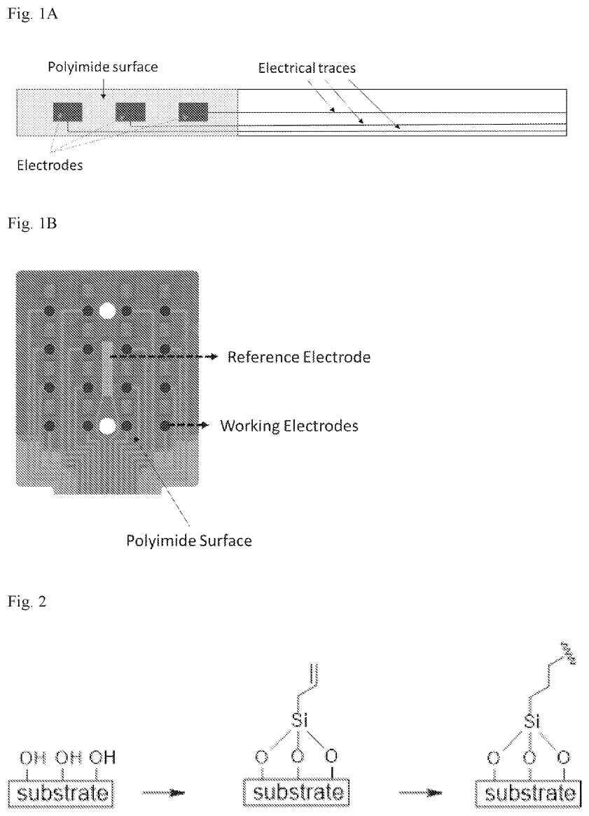

At FIG. 1A, one sees an exemplary sensor system. The distal end, left, is configured to be located under the skin, while the proximal end is configured for electrical connection. The sensor has in this case a thickness of 50μ to 150μ. The sensor comprises three electrodes: a working electrode, left, of sputter coated Pt, a central reference electrode of Ag/AgCl ink, and a rightmost counter electrode of sputter coated Pt, used to generate a bias voltage. Traces emerge from each electrode for electrical connections.

At FIG. 1B, one sees an exemplary sensor system having multiple working electrodes and a reference electrode on a polyimide surface.

At FIG. 2, one sees addition of a tie layer to a functionalized surface.

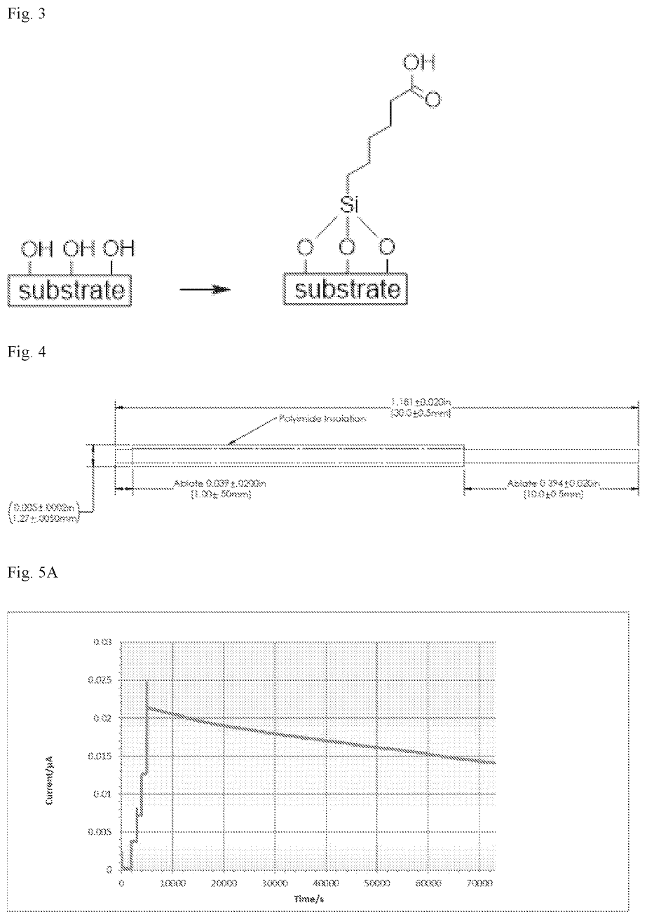

At FIG. 3, one sees addition of a tie layer to a functionalized surface.

At FIG. 4, one sees a schematic of a wire probe core. Only the solid core and the insulating membrane are shown. The solid base is coated by the polyimide insulation layer by dipping into a pre-polymerized imide solution. Following polymerization, the outermost 1 mm of the solid base at left is ablated so as to expose the conductive solid base. The leftmost end of the probe is exposed by ablating the insulating layer so as to generate an exterior edge rather than by ablating an interior hole in the insulation layer that is not in contact with an exterior edge of the insulating layer. The rightmost end of the probe is also ablated; this portion of the solid base does not contact the sample but conducts current to a measuring device.

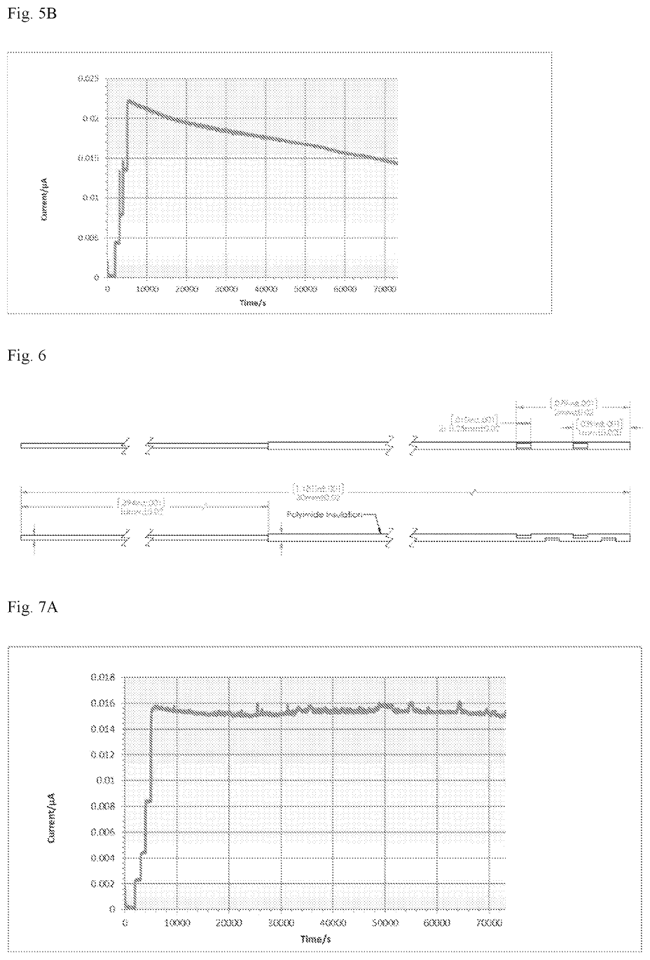

At FIG. 5A and again at Figure SB, one sees a measure of performance of a probe having a core as depicted in FIG. 4. The y axis indicates current in microAmpers, ranging from 0 to 0.03, with 0.005 intervals labeled. The x axis indicates time in seconds, ranging from 0 to 70,000, with 10,000 second intervals marked.

Measurements were made at 400 mg/dL glucose, and drift absolute values of 2. 2% and 1. 9% were observed in Figure SA and FIG. 5B, respectively.

At FIG. 6, one sees a schematic of a wire probe core. Only the solid core and the insulating membrane are shown. The solid base is coated by the polyimide insulation layer by dipping into a pre-polymerized imide solution. Following polymerization, four separate 0. 25 mm holes, two on each side, are ablated so as to expose the conductive solid base. The insulating layer is ablated at interior points so as to generate four interior holes in the insulation layer that are not in contact with an exterior edge of the insulating layer. The leftmost end of the probe is also ablated; this portion of the solid base does not contact the sample but conducts current to a measuring device.

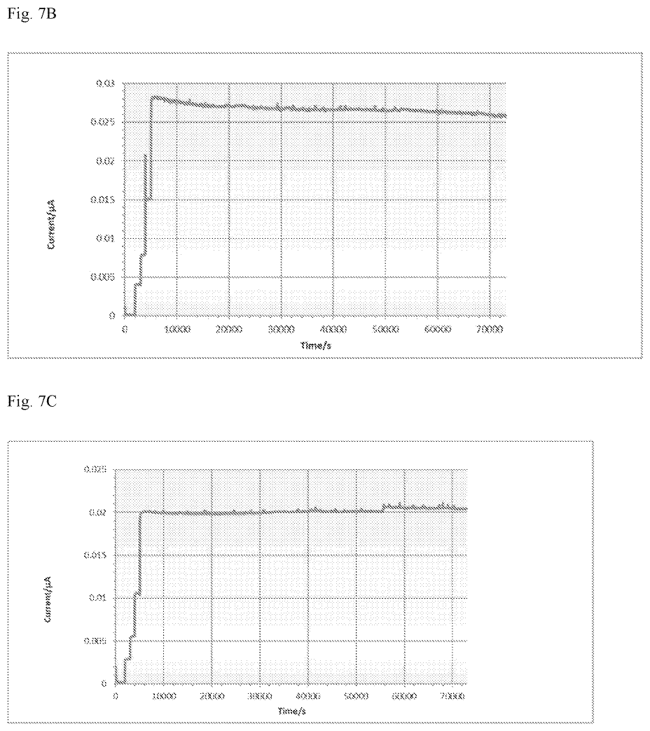

At FIG. 7A, FIG. 7B and FIG. 7C, one sees a measure of performance of a probe having a core as depicted in FIG. 6. The y axis indicates current in microAmpers, ranging from 0 to 0.03, with 0.005 intervals labeled. The x axis indicates time in seconds, ranging from 0 to 70,000, with 10,000 second intervals marked.

Measurements were made at 400 mg/dL glucose, and drift absolute values of 0.2%, 0.5% and 0.1% were observed in FIG. 7A, FIG. 7B and FIG. 7C, respectively.

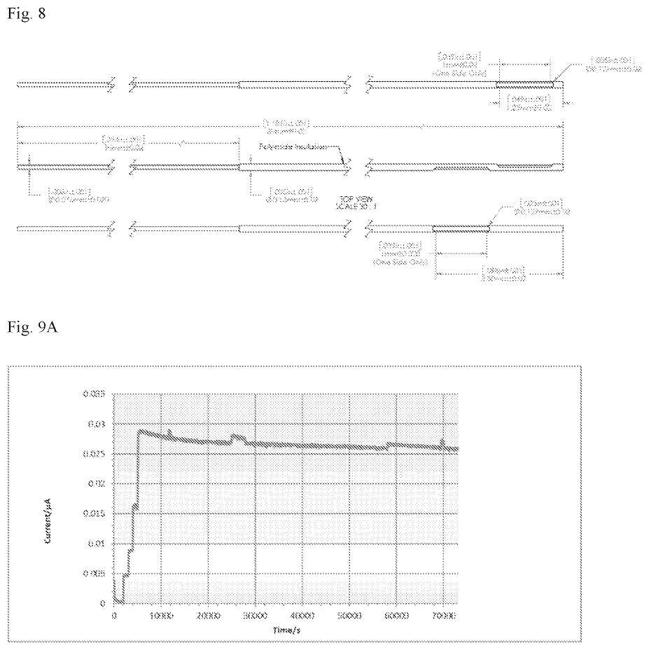

At FIG. 8, one sees a schematic of a wire probe core. Only the solid core and the insulating membrane are shown. The solid base is coated by the polyimide insulation layer by dipping into a pre-polymerized imide solution. Following polymerization, two separate 1 mm holes, one on each side, are ablated so as to expose the conductive solid base. The insulating layer is ablated at interior points so as to generate two interior holes in the insulation layer that are not in contact with an exterior edge of the insulating layer. The leftmost end of the probe is also ablated; this portion of the solid base does not contact the sample but conducts current to a measuring device.

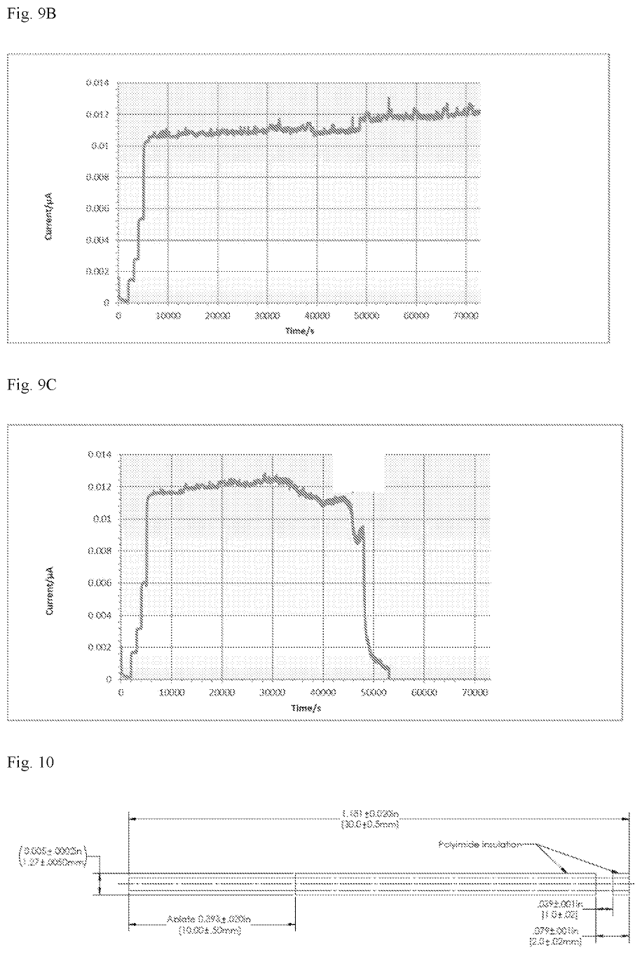

At FIG. 9A, FIG. 9B and FIG. 9C, one sees a measure of performance of a probe having a core as depicted in FIG. 8. The y axis indicates current in microAmpers, ranging from 0 to 0. 03, with 0.005 intervals labeled. The x axis indicates time in seconds, ranging from 0 to 70,000, with 10,000 second intervals marked.

Measurements were made at 400 mg/dL glucose, and drift absolute values of 0.6%, 0.8% and 0.1% were observed in FIG. 9A, FIG. 9B and FIG. 9C, respectively. At FIG. 9C, measurements were impacted by sample evaporation starting after 30,000 seconds, and measurement failed after 45,000 seconds.

At FIG. 10, one sees a schematic of a wire probe core. Only the solid core and the insulating membrane are shown. The solid base is coated by the polyimide insulation layer by dipping into a pre-polymerized imide solution. Following polymerization, a single 1 mm skive, is ablated so as to expose a ring or band of 1 mm in width on the conductive solid base. The insulating layer is ablated so as to generate a band gap in the insulation layer, effectively generating two new exterior edges of the insulating layer, which is now bisected. The leftmost end of the probe is also ablated; this portion of the solid base does not contact the sample but conducts current to a measuring device.

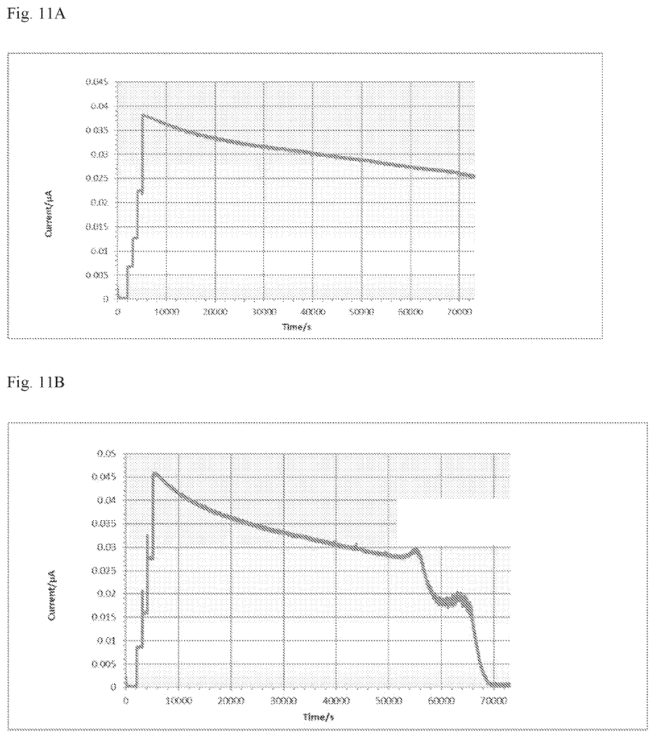

At FIG. 11A and FIG. 11B, one sees a measure of performance of a probe having a core as depicted in FIG. 10. The y axis indicates current in microAmpers, ranging from 0 to 0. 03, with 0.005 intervals labeled. The x axis indicates time in seconds, ranging from 0 to 70,000, with 10,000 second intervals marked.

Measurements were made at 400 mg/dL glucose, and drift absolute values of 1.7% and 3.0% were observed in FIG. 11A and FIG. 11B, respectively. At FIG. 11B, measurements were impacted by sample evaporation starting after 50,000 seconds, and failed shortly thereafter.

Collectively, FIGS. 1 through 8B indicate that the probes tested herein each performed well in long-term glucose assays. However, there was a notable improvement in probe stability, as indicated by lower absolute drift values, in assays using the probe cores of FIG. 6 and FIG. 8, both of which involved probes comprising at least one hole ablated internal to the insulating layer, such that the hole or holes were separate from the external edges of the insulating layer.

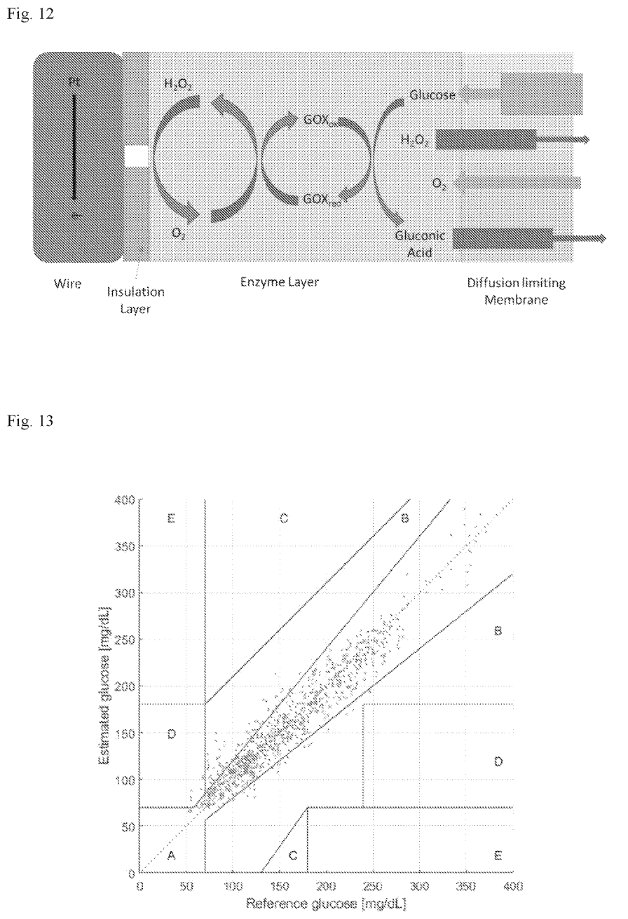

At FIG. 12, we see a schematic of a sensor. The schematic shows a cross-section of the various parts of a glucose detecting sensor system, described as follows.

At left is a platinum sold base that is reactive with hydrogen peroxide and conductive.

To its right is an insulation layer having a hole in its center. The insulation layer is not otherwise porous to hydrogen peroxide, the detection molecule in this system. Hydrogen peroxide that comes into contact with the platinum base reacts to yield a current in the base.

To the right of the insulation layer is the sensing layer, which comprises a biocompatible polymer and a detection moiety, in this case glucose oxidase (GOX). The sensing layer is permeable to hydrogen peroxide, oxygen and glucose, as well as to the solvent or carrier. Glucose that comes into contact with GOX is oxidized to gluconic acid, yielding reduced GOX and hydrogen peroxide. When hydrogen peroxide contacts the reactive platinum base layer, it yields a charge and molecular oxygen, which reacts with reduced GOX to recover oxidized GOX.

To the right of the sensing layer is the semipermeable external membrane. The external membrane is permeable to glucose and molecular oxygen, and semipermeable to gluconic acid and hydrogen peroxide, such that gluconic acid and hydrogen peroxide are allowed to escape but not to enter the regions to the left of the external membrane. As a result, only hydrogen peroxide resulting from GOX activity on glucose is available to react with the platinum at the opening of the insulating layer to generate a current, such that the current is indicative of the concentration of glucose in the solvent or carrier.

The insulating layer, the sensing layer and the external membrane are each covalently linked to their adjacent later so as to maintain the stability of the sensor generally and the integrity of the gap in the insulating layer in particular.

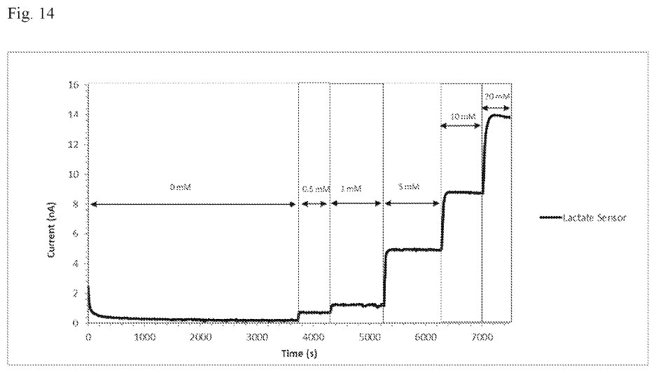

At FIG. 13, one sees a Clark Error Grid of a comparison of a commercial CGM to a 4-hole slit wire configuration as disclosed herein. The y axis indicates estimated glucose, in mg/dL, in units of 50 ranging from 0 to 400. The x axis indicates reference glucose, in mg/dL, in units of 50 ranging from 0 to 400. Region A on the grid indicates points corresponding to measurements within 20% of the reference. Region B indicates points outside of 20% but which would not lead to inappropriate treatment. Region C indicates points leading to inappropriate treatment. Region D indicates a potentially dangerous failure to detect hypoglycemia or hyperglycemia. Region E indicates points that may confuse hyperglycemia and hyperglycemia.

The vast majority of the points fall within region A, indicative of measurements within 20% of the reference, with the remainder largely falling within region B immediately adjacent to region A.

At FIG. 14, one sees performance of a lactate sensor consistent with the disclosure herein. At the y-axis on sees current, in nA, ranging from 0 to 16 in intervals of 2. Along the y-axis one sees time, in seconds, ranging from 0 to 7,000 in intervals of 1,000. This data indicates that lactate, and other analytes aside from glucose are readily sensed using systems disclosed herein, and that lactate oxidase, or a range of enzymes, may be used to initiate a current upon reaction with an analyte.

As used herein, the term “about” in reference to a number refers to a range spanning +/−10% of that number, while in reference to a range, the term refers to an extended range spanning from 10% below the lower limit to 10% above the upper limit of the range.

The disclosure is further understood in light of the following numbered embodiments. 1. A sensor comprising i) a solid base; ii) an insulating membrane affixed to the solid base, wherein the insulating membrane comprises at least one opening that does not share a border with an exterior edge of the insulating layer, so as to expose a portion of the solid base removed from the exterior edge of the insulating layer; iii) a sensor layer comprising a biocompatible polymer and an analyte detection moiety capable of detecting an analyte; and iv) an exterior layer that completely envelops the sensor layer. 2. The sensor of any of the previous embodiments, such as embodiment 1, wherein the solid base is a wire. 3. The sensor of any of the previous embodiments, such as embodiment 1, wherein the solid base comprises a locally planar surface. 4. The sensor of any of the previous embodiments, such as embodiment 1, wherein the solid base can conduct an electric current. 5. The sensor of any of the previous embodiments, such as embodiment 1, wherein the solid base comprises a metal. 6. The sensor of any of the previous embodiments, such as embodiment 5, wherein the metal comprises platinum. 7. The sensor of any of the previous embodiments, such as embodiment 5, wherein the metal comprises rhodium. 8. The sensor of any of the previous embodiments, such as embodiment 5, wherein the metal comprises iridium. 9. The sensor of any of the previous embodiments, such as embodiment 1, wherein the insulating membrane cannot conduct an electric current. 10. The sensor of any of the previous embodiments, such as embodiment 1, wherein the insulating membrane is impermeable to an analyte assayed by the analyte detection moiety. 11. The sensor of any of the previous embodiments, such as embodiment 1, wherein the at least one opening of the insulating membrane is formed via ablation of an unperforated precursor contacted to the solid base. 12. The sensor of any of the previous embodiments, such as embodiment 1, wherein the insulating membrane comprises a plastic. 13. The sensor of any of the previous embodiments, such as embodiment 12, wherein the plastic comprises polyimide. 14. The sensor of any of the previous embodiments, such as embodiment 12, wherein the plastic comprises polyimide selected from the group consisting of polyurethane, PEEK, and Teflon. 15. The sensor of any of the previous embodiments, such as embodiment 1, wherein the insulating layer does not completely envelop the solid base. 16. The sensor of any of the previous embodiments, such as embodiment 1, wherein the insulating layer comprises an insulating layer functionalized reactive moiety. 17. The sensor of any of the previous embodiments, such as embodiment 16, wherein the insulating layer functionalized reactive moiety comprises a hydroxyl moiety. 18. The sensor of any of the previous embodiments, such as embodiment 16, wherein the insulating layer functionalized reactive moiety comprises a carboxyl moiety. 19. The sensor of any of the previous embodiments, such as embodiment 1, wherein the biocompatible polymer of the sensor layer comprises a negatively charged hydrogel. 20. The sensor of any of the previous embodiments, such as embodiment 19, wherein the negatively charged hydrogel comprises polyacrylate, 21. The sensor of any of the previous embodiments, such as embodiment 19, wherein the negatively charged hydrogel comprises polyurethane. 22. The sensor of any of the previous embodiments, such as embodiment 1, wherein the sensor layer is permeable to the analyte. 23. The sensor of any of the previous embodiments, such as embodiment 1, wherein the sensor layer does not completely envelop the insulating membrane. 24. The sensor of any of the previous embodiments, such as embodiment 1, wherein the analyte detection moiety is embedded in the biocompatible polymer. 25. The sensor of any of the previous embodiments, such as embodiment 1, wherein the analyte detection moiety is a protein. 26. The sensor of any of the previous embodiments, such as embodiment 1, wherein the analyte detection moiety is an enzyme. 27. The sensor of any of the previous embodiments, such as embodiment 1, wherein the analyte detection moiety is an antibody. 28. The sensor of any of the previous embodiments, such as embodiment 26, wherein the enzyme is glucose oxidase. 29. The sensor of any of the previous embodiments, such as embodiment 26, wherein the enzyme is lactate oxidase. 30. The sensor of any of the previous embodiments, such as embodiment 26, wherein the enzyme is an oxidase. 31. The sensor of any of the previous embodiments, such as embodiment 26, wherein the enzyme yields hydrogen peroxide when in contact with the analyte. 32. The sensor of any of the previous embodiments, such as embodiment 1, wherein the analyte detection moiety reacts with the analyte to form a detection product. 33. The sensor of any of the previous embodiments, such as embodiment 32, wherein the detection product yields a current when in contact with the solid base. 34. The sensor of any of the previous embodiments, such as embodiment 32, wherein the detection product comprises hydrogen peroxide. 35. The sensor of any of the previous embodiments, such as embodiment 32, wherein the detection product comprises an electric current. 36. The sensor of any of the previous embodiments, such as embodiment 32, wherein the insulating layer is impermeable to the detection product. 37. The sensor of any of the previous embodiments, such as embodiment 32, wherein the sensor layer is permeable to the detection product. 38. The sensor of any of the previous embodiments, such as embodiment 32, wherein the exterior layer is permeable to the detection product. 39. The sensor of any of the previous embodiments, such as embodiment 32, wherein the exterior layer is impermeable to the detection product. 40. The sensor of any of the previous embodiments, such as embodiment 16, wherein the sensor layer comprises a sensor layer functionalized reactive moiety. 41. The sensor of any of the previous embodiments, such as embodiment 40, wherein the sensor layer functionalized reactive moiety comprises a carboxy moiety. 42. The sensor of any of the previous embodiments, such as embodiment 40, wherein the sensor layer functionalized reactive moiety comprises a hydroxy moiety. 43. The sensor of any of the previous embodiments, such as embodiment 40, wherein at least one sensor layer functionalized reactive moiety is covalently bound to at least one insulating membrane functionalized reactive moiety. 44. The sensor of any of the previous embodiments, such as embodiment 1, wherein the exterior layer is permeable to the analyte. 45. The sensor of any of the previous embodiments, such as embodiment 1, wherein the exterior layer is semipermeable to the analyte. 46. The sensor of any of the previous embodiments, such as embodiment 1, wherein the exterior layer limits diffusion of the analyte. 47. The sensor of any of the previous embodiments, such as embodiment 43, wherein the exterior layer comprises an exterior layer functionalized reactive moiety. 48. The sensor of any of the previous embodiments, such as embodiment 47, wherein the exterior layer functionalized reactive moiety comprises a vinyl moiety. 49. The sensor of any of the previous embodiments, such as embodiment 47, wherein the exterior layer functionalized reactive moiety comprises a hydride moiety. 50. The sensor of any of the previous embodiments, such as embodiment 47, wherein at least one exterior layer functionalized reactive moiety is covalently bound to at least one sensing membrane functionalized reactive moiety. 51. The sensor of any of the previous embodiments, such as embodiment 1, wherein the exterior layer comprises an exterior layer functionalized reactive moiety. 52. The sensor of any of the previous embodiments, such as embodiment 51, wherein the exterior layer functionalized reactive moiety comprises a vinyl moiety. 53. The sensor of any of the previous embodiments, such as embodiment 51, wherein the exterior layer functionalized reactive moiety comprises a hydride moiety. 54. The sensor of any of the previous embodiments, such as embodiment 51, wherein at least one exterior layer functionalized reactive moiety is covalently bound to at least one functionalized reactive moiety on the sensing layer. 55. The sensor of any of the previous embodiments, such as embodiment 1, wherein the at least one opening comprises at least two openings. 56. The sensor of any of the previous embodiments, such as embodiment 1, wherein the at least one exterior opening comprises at least three openings. 57. The sensor of any of the previous embodiments, such as embodiment 1, wherein the at least one exterior opening comprises at least four openings. 58. The sensor of any of the previous embodiments, such as embodiment 1, wherein the sensor exhibits a signal drift of no more than 1% of signal/hour. 59. The sensor of any of the previous embodiments, such as embodiment 58, wherein the sensor exhibits a signal drift of no more than 1% of signal/hour after four days of monitoring. 60. A method of making a sensor, the method comprising contacting a solid base to an insulating agent to form an insulating layer on the solid base; forming at least one hole in the insulating layer such that the solid base is exposed at the hole; contacting the insulating layer to a detection precursor comprising a biocompatible polymer precursor and an analyte detection moiety capable of detecting an analyte; polymerizing the biocompatible polymer precursor to form a sensor layer that spans at least part of the insulating layer and the at least one hole in the insulating layer; crosslinking at least a portion of the sensor layer to at least a portion of the insulating layer; contacting the sensor layer to an exterior layer agent to form an exterior layer, and crosslinking at least a portion of the sensor layer to at least a portion of the exterior layer. 61. The method of any of the previous embodiments, such as embodiment 60, wherein contacting the solid base to the insulating agent comprises spraying the insulating agent onto a flat surface of the solid base. 62. The method of any of the previous embodiments, such as embodiment 60, wherein contacting the solid base to the insulating agent comprises dipping the solid base into a liquid composition of the insulating agent. 63. The method of any of the previous embodiments, such as embodiment 62, wherein the solid base is locally cylindrical. 64. The method of any of the previous embodiments, such as embodiment 60, wherein the solid base is an electrical conductor. 65. The method of any of the previous embodiments, such as embodiment 64, wherein the solid base is a metal. 66. The method of any of the previous embodiments, such as embodiment 65, wherein the solid base comprises platinum. 67. The method of any of the previous embodiments, such as embodiment 65, wherein the solid base comprises rhodium. 68. The method of any of the previous embodiments, such as embodiment 65, wherein the solid base comprises iridium. 69. The method of any of the previous embodiments, such as embodiment 60, wherein the insulating layer is not an electrical conductor. 70. The method of any of the previous embodiments, such as embodiment 60, wherein the insulating layer is impermeable to the analyte. 71. The method of any of the previous embodiments, such as embodiment 60, wherein the insulating layer is impermeable to a solvent. 72. The method of any of the previous embodiments, such as embodiment 60, wherein the insulating layer comprises a polymer. 73. The method of any of the previous embodiments, such as embodiment 60, wherein the insulating layer comprises a plastic. 74. The method of any of the previous embodiments, such as embodiment 60, wherein the insulating layer comprises polyimide. 75. The method of any of the previous embodiments, such as embodiment 60, wherein the insulating layer is a polymerization product. 76. The method of any of the previous embodiments, such as embodiment 60, wherein the insulating layer is a solidified product. 77. The method of any of the previous embodiments, such as embodiment 60, wherein forming at least one hole in the insulating layer comprises etching. 78. The method of any of the previous embodiments, such as embodiment 77, wherein the etching is chemical etching. 79. The method of any of the previous embodiments, such as embodiment 77, wherein the etching is laser etching. 80. The method of any of the previous embodiments, such as embodiment 77, wherein the etching is mechanical etching. 81. The method of any of the previous embodiments, such as embodiment 60, wherein forming at least one hole in the insulating layer comprises ablation. 82. The method of any of the previous embodiments, such as embodiment 60, wherein the solid base is accessible to a solvent at the at least one hole. 83. The method of any of the previous embodiments, such as embodiment 60, wherein the insulating agent comprises a functionalizing moiety. 84. The method of any of the previous embodiments, such as embodiment 60, wherein the insulating layer comprises an insulating layer functionalizing moiety. 85. The method of any of the previous embodiments, such as embodiment 84, wherein the insulating layer functionalizing moiety comprises a hydroxy moiety. 86. The method of any of the previous embodiments, such as embodiment 84, wherein the insulating layer functionalizing moiety comprises a carboxy moiety. 87. The method of any of the previous embodiments, such as embodiment 60, wherein the insulating layer is modified to comprise a functionalizing moiety prior to contacting the insulating layer to the detection precursor. 88. The method of any of the previous embodiments, such as embodiment 60, wherein the insulating layer is modified to comprise a functionalizing moiety concurrently to contacting the insulating layer to the detection precursor. 89. The method of any of the previous embodiments, such as embodiment 60, wherein the insulating layer is modified to comprise a functionalizing moiety subsequent to contacting the insulating layer to the detection precursor 90. The method of any of the previous embodiments, such as embodiment 60, wherein the detection precursor is a liquid. 91. The method of any of the previous embodiments, such as embodiment 60, wherein contacting the detection precursor to the insulating layer comprises dipping the solid base and the insulating layer into the detection precursor 92. The method of any of the previous embodiments, such as embodiment 60, wherein contacting the detection precursor to the insulating layer comprises spraying the detection precursor onto the insulating layer. 93. The method of any of the previous embodiments, such as embodiment 60, wherein biocompatible polymer precursor comprises a biocompatible layer functionalizing moiety. 94. The method of any of the previous embodiments, such as embodiment 93, wherein the biocompatible layer functionalizing moiety comprises a vinyl moiety. 95. The method of any of the previous embodiments, such as embodiment 93, wherein the biocompatible layer functionalizing moiety comprises a hydride moiety. 96. The method of any of the previous embodiments, such as embodiment 60, wherein polymerizing the biocompatible polymer precursor to form a sensor layer comprises incorporating at least one of vinyl functional silicone or a hydride functional silicone. 97. The method of any of the previous embodiments, such as embodiment 96, wherein polymerizing comprises incorporating a hydrophilic component and wherein the polymerization yields a semipermeable membrane. 98. The method of any of the previous embodiments, such as embodiment 60, wherein crosslinking at least a portion of the base layer to at least a portion of the insulating layer comprises crosslinking a platinum vinyl group to a hydride group via a hydrosilylation reaction. 99. The method of any of the previous embodiments, such as embodiment 60, wherein contacting the sensor layer to an exterior layer agent comprises depositing the exterior layer agent onto the sensor layer. 100. The method of any of the previous embodiments, such as embodiment 60, wherein contacting the sensor layer to an exterior layer agent comprises dipping the sensor layer into the exterior layer agent. 101. The method of any of the previous embodiments, such as embodiment 60, wherein the exterior layer is polymerized silicone. 102. The method of any of the previous embodiments, such as embodiment 60, wherein the exterior layer comprises an exterior layer functionalizing agent. 103. The method of any of the previous embodiments, such as embodiment 102, wherein the biocompatible layer functionalizing moiety comprises a hydroxyl moiety. 104. The method of any of the previous embodiments, such as embodiment 102, wherein the biocompatible layer functionalizing moiety comprises a carboxy moiety. 105. A glucose sensor that exhibits a signal drift of no more than 1% of signal/hour. 106. The glucose sensor of any of the previous embodiments, such as embodiment 105, wherein signal drift is assayed at least four days into a testing regimen. 107. The glucose sensor of any of the previous embodiments, such as embodiment 105, wherein signal drift is assayed against a concentration of glucose of about 400 mg/dL. 108. The glucose sensor of any of the previous embodiments, such as embodiment 105, wherein signal drift is assayed against a concentration of glucose of at least 100 mg/dL. 109. The glucose sensor of any of the previous embodiments, such as embodiment 105, wherein the sensor comprises an insulating layer having a hole distal from an edge of the insulating layer. 110. The glucose sensor of any of the previous embodiments, such as embodiment 109, wherein the sensor comprises a sensor membrane covalently bound to the insulating layer. 111. The glucose sensor of any of the previous embodiments, such as embodiment 109, wherein the sensor comprises a sensor membrane covalently bound to an exterior layer. 112. A method of extending sensor performance, comprising affixing a sensor membrane to a sensor core, and binding an exterior coating to the sensor membrane, so as to prevent at least some of the sensor membrane from separating from the sensor core upon introducing a liquid to a sensor. 113. The method of any of the previous embodiments, such as embodiment 112, wherein the binding is noncovalent binding. 114. The method of any of the previous embodiments, such as embodiment 112, wherein the binding is covalent binding. 115. The method of any of the previous embodiments, such as embodiment 112, wherein the sensor exhibits a signal drift of no more than 1% of signal/hour. 116. The method of any of the previous embodiments, such as embodiment 115, wherein signal drift is assayed at least four days into a testing regimen. 117. The method of any of the previous embodiments, such as embodiment 115, wherein signal drift is assayed against a concentration of ana analyte of about 400 mg/dL. 118. The method of any of the previous embodiments, such as embodiment 115, wherein signal drift is assayed against a concentration of ana analyte of at least 100 mg/dL. 119. A method of detecting an analyte in a sample, comprising creating a voltage bias in the sample, and measuring a current passing to a conductive base through at least one opening in an insulating layer, wherein the at least one opening does not share a border with an exterior edge of the insulating layer. 120. The method of any of the previous embodiments, such as embodiment 119, wherein the sample is a body fluid. 121. The method of any of the previous embodiments, such as embodiment 119, wherein the detecting is performed on a fluid internal to an individual. 122. The method of any of the previous embodiments, such as embodiment 119, wherein the voltage bias is no more than 1 volt. 123. The method of any of the previous embodiments, such as embodiment 119, wherein the voltage bias is no more than 0.6 volt. 124. The method of any of the previous embodiments, such as embodiment 119, wherein the voltage bias is no more than 0.3 volt. 125. The method of any of the previous embodiments, such as embodiment 119, wherein the current arises from hydrogen peroxide contacting the conductive base. 126. The method of any of the previous embodiments, such as embodiment 119, wherein the current arises from oxidation of the analyte. 127. The method of any of the previous embodiments, such as embodiment 126, wherein the current arises from an enzymatic reaction on the analyte. 128. The method of any of the previous embodiments, such as embodiment 127, wherein the enzymatic reaction comprises action of an oxidase. 129. The method of any of the previous embodiments, such as embodiment 127, wherein the enzymatic reaction comprises action of glucose oxidase. 130. The method of any of the previous embodiments, such as embodiment 127, wherein the enzymatic reaction comprises action of lactate oxidase. 131. The method of any of the previous embodiments, such as embodiment 126, wherein the analyte comprises glucose. 132. The method of any of the previous embodiments, such as embodiment 126, wherein the analyte comprises lactate. 133. The method of any of the previous embodiments, such as embodiment 119, comprising subjecting the analyte to a reaction to generate the current. 134. The method of any of the previous embodiments, such as embodiment 133, wherein the reaction comprises oxidation. 135. The method of any of the previous embodiments, such as embodiment 134, wherein the oxidation comprises enzymatic oxidation. 136. The method of any of the previous embodiments, such as embodiment 135, wherein the oxidation yields a reactive oxygen species. 137. The method of any of the previous embodiments, such as embodiment 135, wherein the oxidation yields hydrogen peroxide. 138. The method of any of the previous embodiments, such as embodiment 137, wherein the oxidation yields the hydrogen peroxide in proportion to the amount of the analyte. 139. The method of any of the previous embodiments, such as embodiment 133, wherein the analyte comprises glucose. 140. The method of any of the previous embodiments, such as embodiment 133, wherein the analyte comprises lactate. 141. The method of any of the previous embodiments, such as embodiment 119, wherein the current has an amplitude that is proportional to concentration of the analyte. 142. The method of any of the previous embodiments, such as embodiment 119, wherein the current has an amplitude that is proportional to amount of the analyte. 143. The method of any of the previous embodiments, such as embodiment 141 or embodiment 142, wherein the analyte comprises glucose. 144. The method of any of the previous embodiments, such as embodiment 141 or embodiment 142, wherein the analyte comprises lactate. 145. The method of any of the previous embodiments, such as embodiment 119, wherein the conductive base directs the current to a detector. 146. The method of any of the previous embodiments, such as embodiment 119, wherein the conductive base comprises a channel that directs the current to a detector. 147. The method of any of the previous embodiments, such as embodiment 119, comprising limiting diffusion of the analyte to the opening. 148. The method of any of the previous embodiments, such as embodiment 147, wherein limiting diffusion comprises covering the opening with an external layer. 149. The method of any of the previous embodiments, such as embodiment 148, wherein the exterior layer is permeable to the analyte. 150. The method of any of the previous embodiments, such as embodiment 148, wherein the exterior layer is semipermeable to the analyte. 151. The method of any of the previous embodiments, such as embodiment 119, wherein the insulating layer precludes current from passing to the conductive base other than through the at least one opening. 152. The method of any one of any of the previous embodiments, such as embodiments 119 to 151, wherein the detecting is performed for at least 2 days. 153. The method of any one of any of the previous embodiments, such as embodiments 119 to 151, wherein the detecting is performed for at least 4 days. 154. The method of any of the previous embodiments, such as embodiment 119, wherein the detecting exhibits a signal drift of no more than 1% of signal/hour. 155. The method of any of the previous embodiments, such as embodiment 119, wherein the detecting exhibits a signal drift of no more than 1% of signal/hour after four days of monitoring. 156. The method of any one of any of the previous embodiments, such as embodiments 119 to 155, wherein the measuring a current passing to a conductive base through at least one opening in an insulating layer, wherein the at least one opening does not share a border with an exterior edge of the insulating layer is performed by a sensor or any one of any of the previous embodiments, such as embodiments 1 to 59.

### Examples

Example 1. Wire sensor configurations were tested as to their effect on sensor stability and accuracy. Wire sensors varied in the degree and configuration of the access to their solid base conductors as provided by their insulating layers. Configurations included 1 mm tip exposure, 2× slit holes, 4× slit boles, and 1× skive internal loop insulation layer removal. All wires comprised a solid core, an insulating membrane, a sensor layer and an external membrane, with crosslinking among layers as disclosed herein.

Wire probes were assayed for accuracy of measurement of a test liquid of 0, 50, 100, 200 and 400 mg/dL glucose, at a voltage bias of 285 m V, for 4 consecutive days. Sensers were inserted into vials of the aforementioned compositions for 15 hours per day to determine drift and stability of measurements, and data were collected for the fourth day of each experiment.

The 1 mm tip exposure sensor configuration is presented in FIG. 4. The left side of the wire is exposed to the test liquid, such that the only portion of the solid base accessible to the test liquid is the leftmost ablated 1 mm tip of the needle. Measurements are presented for runs W1 and W3. Measurement results are presented in Table 2 and Table 3, and in FIG. 5A and FIG. 5B.

TABLE 2
1 mm Tip exposure wire glucose assay results at 4 days.

| glu (mg/dL) | pA W1 | pA W3 |
|---|---|---|
| 0 | 1.31E+02 | 1.13E+02 |
| 50 | 3.82E+03 | 4.24E+03 |
| 100 | 7.18E+03 | 7.88E+03 |
| 200 | 1.27E+04 | 1.35E+04 |
| 400 | 2.45E+04 | 2.23E+04 |
| Sensitivity | 60 | 54 |
| (pA/mg/dL) | | |
| Baseline | 658 | 1491 |
| R² | 0.998 | 0.983 |

Measurements showed an R² value of greater than 0. 98, indicating remarkable linearity of measurements across the range of 0-400 mg/dL.

Drift in measurement values were measured over time for the 15 hour assay at day 4. The results are shown in Table 3 and in FIG. 5A and FIG. 5B.

TABLE 3
Signal Drift - Tip exposure wire.

| Drift at 400 mg/dL glucose | W1 | W3 |
|---|---|---|
| INITIAL uA | 2.45E−02 | 2.23E−02 |
| FINAL uA | 1.41E−02 | 1.43E−02 |
| % Change (Total) | −42.4% | −35.7% |
| % Change (Per hour) | −2.2% | −1.9% |

Measurements showed a drift having an absolute value of about 2% per hour.

The accuracy and stability of the 1 mm exposed tip wire sensor demonstrates the benefit of crosslinked sensor layers on sensor performance, as both the R² value across glucose concentrations and the % change per hour showed impressive results.

The 4× slit hole exposure sensor configuration is presented in FIG. 6. The right side of the wire is exposed to the test liquid, such that the only portions of the solid base accessible to the test liquid are the four portions exposed by the four ablated slit boles in the insulating layer. Measurements are presented for runs W4, W5 and W6. Measurement results are presented in Table 4 and Table 5, and in FIG. 7A, FIG. 7B and FIG. 7C.

TABLE 4
4x slit hole exposure wire glucose assay results at 4 days.

| glu (mg/dL) | pA W4 | pA W5 | pA W6 |
|---|---|---|---|
| 0 | 1.45E+02 | 1.10E+02 | 1.29E+02 |
| 50 | 2.28E+03 | 4.06E+03 | 2.90E+03 |
| 100 | 4.37E+03 | 7.92E+03 | 5.55E+03 |
| 200 | 8.35E+04 | 1.51E+04 | 1.04E+04 |
| 400 | 1.58E+04 | 2.83E+04 | 2.00E+04 |
| Sensitivity | 39 | 70 | 49 |
| (pA/mg/dL) | | | |
| Baseline | 350 | 580 | 391 |
| R² | 0.999 | 0.999 | 0.999 |

Measurements showed an R² value of 0.999, indicating remarkable linearity of measurements across the range of 0-400 mg/dL and a substantial improvement over the 1 mm tip exposure.

Drift in measurement values were measured over time for the 15 hour assay at day 4. The results are shown in Table 5 and in FIGS. 4A-4C.

TABLE 5
Signal Drift - 4x slit hole exposure wire.

| Drift at 400 mg/dL glucose | W4 | W5 | W6 |
|---|---|---|---|
| INITIAL uA | 1.58E−02 | 2.83E−02 | 2.00E−02 |
| FINAL uA | 1.51E−02 | 2.57E−02 | 2.04E−02 |
| % Change (Total) | −4.1% | −9.1% | 1.8% |
| % Change (Per hour) | −0.2% | −0.5% | 0.1% |

Measurements showed a drift having an absolute value of no more than 0.5%, with an average of less than 0.3% per hour.

The accuracy and stability of the 4× slit hole wire sensor demonstrates the benefit of crosslinked sensor layers on sensor performance, as both the R² value across glucose concentrations and the % change per hour showed impressive results. The results further demonstrate the substantial differential improvement of using a sensor probe having a hole internal to an exterior edge of the insulating membrane rather than an exposed edge sensor.

The 2× slit hole exposure sensor configuration is presented in FIG. 8.

The right side of the wire is exposed to the test liquid, such that the only portions of the solid base accessible to the test liquid are the four portions exposed by the two ablated slit holes in the insulating layer. Measurements are presented for runs W7, W8 and W9. Measurement results are presented in Table 6 and Table 7, and in FIG. 9A, FIG. 9B and FIG. 9C.

TABLE 6
2x slit hole exposure wire glucose assay results at 4 days.

| glu (mg/dL) | pA W7 | pA W8 | pA W9 |
|---|---|---|---|
| 0 | 2.40E+02 | 1.49E+02 | 1.58E+02 |
| 50 | 4.68E+03 | 1.42E+03 | 1.70E+03 |
| 100 | 8.78E+03 | 2.82E+03 | 3.19E+03 |
| 200 | 1.58E+04 | 5.38E+04 | 5.83E+04 |
| 400 | 2.58E+04 | 1.05E+04 | 1.16E+04 |
| Sensitivity | 63 | 26 | 28 |
| (pA/mg/dL) | | | |
| Baseline | 1604 | 163 | 241 |
| R² | 0.985 | 1.000 | 1.000 |

Measurements showed an R² value of 0.995, with two of the three runs exhibiting an R² of 1.000, indicating remarkable linearity of measurements across the range of 0-400 mg/dL and a substantial improvement over the 1 mm tip exposure.

Drift in measurement values were measured over time for the 15 hour assay at day 4. The results are shown in Table 7 and in FIGS. 6A-6C.

TABLE 7
Signal Drift - 2x slit hole exposure wire.

| Drift at 400 mg/dL glucose | W7 | W8 | W9 |
|---|---|---|---|
| INITIAL uA | 2.88E−02 | 1.05E−02 | 1.16E−02 |
| FINAL uA | 2.58E−02 | 1.21E−02 | 1.14E−02 |
| % Change (Total) | −10.3% | 15% | −1.5% |
| % Change (Per hour) | −0.6% | 0.8% | −0.1% |

Measurements showed a drift having an absolute value of no more than 0.8%, with an average of than 0.5% per hour.

The accuracy and stability of the 2× slit hole wire sensor demonstrates the benefit of crosslinked sensor layers on sensor performance, as both the R² value across glucose concentrations and the % change per hour showed impressive results. The results further demonstrate the substantial differential improvement of using a sensor probe having a hole internal to an exterior edge of the insulating membrane rather than an exposed edge sensor, as the 2× slit hole wire sensor substantially outperformed the 1 mm exposed end Sensor.

The 1× skived ring exposure sensor configuration is presented in FIG. 10. The right side of the wire is exposed to the test liquid, such that the only portions of the solid base accessible to the test liquid are the four portions exposed by the two ablated slit holes in the insulating layer. Measurements are presented for runs W10 and W12. Measurement results are presented in Table 8 and Table 9, and in FIG. 11A and FIG. 11B.

TABLE 8
1x skived exposure wire glucose assay results at 4 days.

| glu (mg/dL) | pA W10 | pA W12 |
|---|---|---|
| 0 | 7.81E+01 | 2.91E+02 |
| 50 | 6.79E+03 | 8.56E+03 |
| 100 | 1.26E+04 | 1.58E+03 |
| 200 | 2.18E+04 | 2.75E+04 |
| 400 | 3.79E+04 | 4.58E+04 |
| Sensitivity | 92 | 112 |
| (pA/mg/dL) | | |
| Baseline | 1978 | 2786 |
| R² | 0.990 | 0.984 |

Measurements showed an R² value of 0. 987, indicating remarkable linearity of measurements across the range of 0-400 mg/dL.

Drift in measurement values were measured over time for the 15 hour assay at day 4. The results are shown in Table 9 and in FIGS. 6A-6C.

TABLE 9
Signal Drift - 1x skived exposure wire.

| Drift at 400 mg/dL glucose | W10 | W12 |
|---|---|---|
| INITIAL uA | 3.79E−02 | 4.58E−02 |
| FINAL uA | 2.56E−02 | 2.78E−02 |
| % Change (Total) | −32.4% | −39.3% |
| % Change (Per hour) | −1.7% | −3.0% |

Measurements showed a drift having an average absolute value of 2. 35% per hour.

The accuracy and stability of the 1× skived wire sensor demonstrates the benefit of crosslinked sensor layers on sensor performance, as the R² value across glucose concentrations and showed impressive results.

The results further demonstrate the substantial differential improvement of using a sensor probe having a hole internal to an exterior edge of the insulating membrane rather than an exposed edge sensor, as the 2× slit hole wire sensor and the 4× slit hole wire sensor substantially outperformed the 1× skived end sensor and the 1 mm exposed end sensor in terms of sensor measurement drift.

In summary, these results demonstrate the benefit of crosslinking layers on sensor accuracy. This improvement is reflected in the high R² values for all sensors measured.

These results also indicate the increased stability, particularly of sensors in a liquid carrier over prolonged periods of time, of sensors comprising an internal opening such as a hole removed from an exterior edge of the insulation membrane as a solid base access point. This increased stability is reflected in the substantially improved % change per hour of the 2× hole and 4× hole sensor configurations relative to the sensors for which the solid base access area is not a hole.

Example 2. Sensor results of Example 1 were compared to commercially available sensor performance. The results are indicated below.

TABLE 10
Commercially available product assay results.

| | Commercial |
|---|---|
| glu (mg/dL) | Sensor (pA) |
| 0 | 2068 |
| 50 | 2880 |
| 100 | 3700 |
| 200 | 5310 |
| 400 | 8570 |
| Sensitivity | 16 |
| (pA/mg/dL) | |
| Baseline | 2068 |
| R² | 1.00 |

Commercial sensors exhibited comparable sensor accuracy over the range of analyte concentrations tested. The sensitivity and R² demonstrate signal level in particular range; i.e., a sensor with >50 pA/mg/dL might seem like a lot of signal but if the R² (linearity) is poor than that is not a good sensor. The limitation here has to do with oxygen available in the body. The GOX needs oxygen to function and if there is not enough then the reaction becomes limited by oxygen instead of glucose and a non-linear response is obtained.

TABLE 11
Signal drift - commercially available product.

| Drift at 400 mg/dL glucose | W10 |
|---|---|
| INITIAL uA | 8.57E−03 |
| FINAL uA | 7.74E−03 |
| % Change (Total) | −32.4% |
| % Change (Per hour) | −7.3% |

The commercial sensor exhibited a drift per hour having an absolute value of over 7%, which is 2-3 fold higher than even the highest drift values observed for the stabilized sensors of the present disclosure, and is 10-20× higher than the drift values observed for sensors having insulation layers for which there was an internal ablated access area to the solid base. These results were observed despite the assay being performed over a substantially shorter time span than that of the assays in Example 1.

This example illustrates the improvements, individually and in combination, of crosslinking sensor layers or membranes to stabilize sensor structure, and of the use of an internal ablated access area to stabilize the solid base access area, in improving sensor performance by reducing signal drift.

Example 3. Insulation Layer Deposition Insulation layer is deposited onto a solid base pursuant to generation of a molecular sensor as disclosed herein. A solution of polyimide precursor (such as polyamic acid), is dip-coated onto the substrate via a reel-to-reel system and then heat is applied to remove solvent and solidify the polymer on the surface of the base metal as a thin coating. The wire is 0.0033″ and with coating the diameter becomes 0.004″ to 0.005″.

Example 4. A sensor made with the 4-hole slit wire configuration was tested in vivo and compared to a commercial CGM as a reference. The study measured glucose continuously over 10 days and gave a 10% MARD. The Clark-Error Grid is given in FIG. 13.

Example 5. A lactate sensor comprising a solid core, an insulating membrane, a sensor layer and an external membrane, with crosslinking among layers as disclosed herein, was prepared to demonstrate the generality of the sensing platform. The sensor had a sensor layer comprising lactate oxidase and measured various concentrations of sodium lactate (0, 0.5 mM, 1 mM, 5 mM, 10 mM, and 20 mM) in PBS solution. The sensing profile is given in FIG. 14.

The disclosure is further understood in light of the listing of the claims, below.

We claim:

1. A sensor comprising
i) a solid base;
ii) an insulating membrane affixed to the solid base, wherein the insulating membrane comprises at least one opening that does not share a border with an exterior edge of the insulating layer, so as to expose a portion of the solid base removed from the exterior edge of the insulating layer;
iii) a sensor layer comprising a biocompatible polymer and an analyte detection moiety capable of detecting an analyte; and
iv) an exterior layer that completely envelops the sensor layer.

2. The sensor of claim 1, wherein the solid base is a wire.

3. The sensor of claim 1, wherein the solid base comprises a locally planar surface.

4. The sensor of claim 1, wherein the solid base can conduct an electric current.

5. The sensor of claim 1, wherein the solid base comprises a metal.

6. The sensor of claim 5, wherein the metal comprises platinum.

7. The sensor of claim 5, wherein the metal comprises rhodium.

8. The sensor of claim 5, wherein the metal comprises iridium.

9. The sensor of claim 1, wherein the insulating membrane cannot conduct an electric current.

10. The sensor of claim 1, wherein the insulating membrane is impermeable to an analyte assayed by the analyte detection moiety.

11. The sensor of claim 1, wherein the at least one opening of the insulating membrane is formed via ablation of an unperforated precursor contacted to the solid base.

12. The sensor of claim 1, wherein the insulating membrane comprises a plastic.

13. The sensor of claim 12, wherein the plastic comprises polyimide.

14. The sensor of claim 12, wherein the plastic comprises polyimide selected from the group consisting of polyurethane, PEEK, and Teflon.

15. The sensor of claim 1, wherein the insulating layer does not completely envelop the solid base.

16. The sensor of claim 1, wherein the insulating layer comprises an insulating layer functionalized reactive moiety.

17. The sensor of claim 16, wherein the insulating layer functionalized reactive moiety comprises a hydroxyl moiety.

18. The sensor of claim 16, wherein the insulating layer functionalized reactive moiety comprises a carboxyl moiety.

19. The sensor of claim 1, wherein the biocompatible polymer of the sensor layer comprises a negatively charged hydrogel.

20. The sensor of claim 19, wherein the negatively charged hydrogel comprises polyacrylate.

21. The sensor of claim 19, wherein the negatively charged hydrogel comprises polyurethane.

22. The sensor of claim 1, wherein the sensor layer is permeable to the analyte.

23. The sensor of claim 1, wherein the sensor layer does not completely envelop the insulating membrane.

24. The sensor of claim 1, wherein the analyte detection moiety is embedded in the biocompatible polymer.

25. The sensor of claim 1, wherein the analyte detection moiety is a protein.

26. The sensor of claim 1, wherein the analyte detection moiety is an enzyme.

27. The sensor of claim 1, wherein the analyte detection moiety is an antibody.

28. The sensor of claim 26, wherein the enzyme is glucose oxidase.

29. The sensor of claim 26, wherein the enzyme is lactate oxidase.

30. The sensor of claim 26, wherein the enzyme is an oxidase.

31. The sensor of claim 26, wherein the enzyme yields hydrogen peroxide when in contact with the analyte.

32. The sensor of claim 1, wherein the analyte detection moiety reacts with the analyte to form a detection product

33. The sensor of claim 32, wherein the detection product yields a current when in contact with the solid base.

34. The sensor of claim 32, wherein the detection product comprises hydrogen peroxide.

35. The sensor of claim 32, wherein the detection product comprises an electric current.

36. The sensor of claim 32, wherein the insulating layer is impermeable to the detection product.

37. The sensor of claim 32, wherein the sensor layer is permeable to the detection product.

38. The sensor of claim 32, wherein the exterior layer is permeable to the detection product.

39. The sensor of claim 32, wherein the exterior layer is impermeable to the detection product.

40. The sensor of claim 16, wherein the sensor layer comprises a sensor layer functionalized reactive moiety.

41. The sensor of claim 40, wherein the sensor layer functionalized reactive moiety comprises a carboxy moiety.

42. The sensor of claim 40, wherein the sensor layer functionalized reactive moiety comprises a hydroxy moiety.

43. The sensor of claim 40, wherein at least one sensor layer functionalized reactive moiety is covalently bound to at least one insulating membrane functionalized reactive moiety.

44. The sensor of claim 1, wherein the exterior layer is permeable to the analyte.

45. The sensor of claim 1, wherein the exterior layer is semipermeable to the analyte.

46. The sensor of claim 1, wherein the exterior layer limits diffusion of the analyte.

47. The sensor of claim 43, wherein the exterior layer comprises an exterior layer functionalized reactive moiety.

48. The sensor of claim 47, wherein the exterior layer functionalized reactive moiety comprises a vinyl moiety.

49. The sensor of claim 47, wherein the exterior layer functionalized reactive moiety comprises a hydride moiety.

50. The sensor of claim 47, wherein at least one exterior layer functionalized reactive moiety is covalently bound to at least one sensing membrane functionalized reactive moiety.

51. The sensor of claim 1, wherein the exterior layer comprises an exterior layer functionalized reactive moiety.

52. The sensor of claim 51, wherein the exterior layer functionalized reactive moiety comprises a vinyl moiety.

53. The sensor of claim 51, wherein the exterior layer functionalized reactive moiety comprises a hydride moiety.

54. The sensor of claim 51, wherein at least one exterior layer functionalized reactive moiety is covalently bound to at least one functionalized reactive moiety on the sensing layer.

55. The sensor of claim 1, wherein the at least one opening comprises at least two openings.

56. The sensor of claim 1, wherein the at least one exterior opening comprises at least three openings.

57. The sensor of claim 1, wherein the at least one exterior opening comprises at least four openings.

58. The sensor of claim 1, wherein the sensor exhibits a signal drift of no more than 1% of signal/hour.

59. The sensor of claim 58, wherein the sensor exhibits a signal drift of no more than 1% of signal/hour after four days of monitoring.

60. A method of making a sensor, the method comprising contacting a solid base to an insulating agent to form an insulating layer on the solid base; forming at least one hole in the insulating layer such that the solid base is exposed at the hole; contacting the insulating layer to a detection precursor comprising a biocompatible polymer precursor and an analyte detection moiety capable of detecting an analyte; polymerizing the biocompatible polymer precursor to form a sensor layer that spans at least part of the insulating layer and the at least one hole in the insulating layer; crosslinking at least a portion of the sensor layer to at least a portion of the insulating layer; contacting the sensor layer to an exterior layer agent to form an exterior layer, and crosslinking at least a portion of the sensor layer to at least a portion of the exterior layer.

61. The method of claim 60, wherein contacting the solid base to the insulating agent comprises spraying the insulating agent onto a flat surface of the solid base.

62. The method of claim 60, wherein contacting the solid base to the insulating agent comprises dipping the solid base into a liquid composition of the insulating agent.

63. The method of claim 62, wherein the solid base is locally cylindrical.

64. The method of claim 60, wherein the solid base is an electrical conductor.

65. The method of claim 64, wherein the solid base is a metal.

66. The method of claim 65, wherein the solid base comprises platinum.

67. The method of claim 65, wherein the solid base comprises rhodium.

68. The method of claim 65, wherein the solid base comprises iridium.

69. The method of claim 60, wherein the insulating layer is not an electrical conductor.

70. The method of claim 60, wherein the insulating layer is impermeable to the analyte.

71. The method of claim 60, wherein the insulating layer is impermeable to a solvent.

72. The method of claim 60, wherein the insulating layer comprises a polymer.

73. The method of claim 60, wherein the insulating layer comprises a plastic.

74. The method of claim 60, wherein the insulating layer comprises polyimide.

75. The method of claim 60, wherein the insulating layer is a polymerization product.

76. The method of claim 60, wherein the insulating layer is a solidified product.

77. The method of claim 60, wherein forming at least one hole in the insulating layer comprises etching.

78. The method of claim 77, wherein the etching is chemical etching.

79. The method of claim 77, wherein the etching is laser etching.

80. The method of claim 77, wherein the etching is mechanical etching.

81. The method of claim 60, wherein forming at least one hole in the insulating layer comprises ablation.

82. The method of claim 60, wherein the solid base is accessible to a solvent at the at least one hole.

83. The method of claim 60, wherein the insulating agent comprises a functionalizing moiety.

84. The method of claim 60, wherein the insulating layer comprises an insulating layer functionalizing moiety.

85. The method of claim 84, wherein the insulating layer functionalizing moiety comprises a hydroxy moiety.

86. The method of claim 84, wherein the insulating layer functionalizing moiety comprises a carboxy moiety.

87. The method of claim 60, wherein the insulating layer is modified to comprise a functionalizing moiety prior to contacting the insulating layer to the detection precursor.

88. The method of claim 60, wherein the insulating layer is modified to comprise a functionalizing moiety concurrently to contacting the insulating layer to the detection precursor.

89. The method of claim 60, wherein the insulating layer is modified to comprise a functionalizing moiety subsequent to contacting the insulating layer to the detection precursor

90. The method of claim 60, wherein the detection precursor is a liquid.

91. The method of claim 60, wherein contacting the detection precursor to the insulating layer comprises dipping the solid base and the insulating layer into the detection precursor.

92. The method of claim 60, wherein contacting the detection precursor to the insulating layer comprises spraying the detection precursor onto the insulating layer.

93. The method of claim 60, wherein biocompatible polymer precursor comprises a biocompatible layer functionalizing moiety.

94. The method of claim 93, wherein the biocompatible layer functionalizing moiety comprises a vinyl moiety.

95. The method of claim 93, wherein the biocompatible layer functionalizing moiety comprises a hydride moiety.

96. The method of claim 60, wherein polymerizing the biocompatible polymer precursor to form a sensor layer comprises incorporating at least one of vinyl functional silicone or a hydride functional silicone.

97. The method of claim 96, wherein polymerizing comprises incorporating a hydrophilic component and wherein the polymerization yields a semipermeable membrane.

98. The method of claim 60, wherein crosslinking at least a portion of the base layer to at least a portion of the insulating layer comprises crosslinking a platinum vinyl group to a hydride group via a hydrosilylation reaction.

99. The method of claim 60, wherein contacting the sensor layer to an exterior layer agent comprises depositing the exterior layer agent onto the sensor layer.

100. The method of claim 60, wherein contacting the sensor layer to an exterior layer agent comprises dipping the sensor layer into the exterior layer agent.

101. The method of claim 60, wherein the exterior layer is polymerized silicone.

102. The method of claim 60, wherein the exterior layer comprises an exterior layer functionalizing agent.

103. The method of claim 102, wherein the biocompatible layer functionalizing moiety comprises a hydroxyl moiety.

104. The method of claim 102, wherein the biocompatible layer functionalizing moiety comprises a carboxy moiety.

105. A glucose sensor that exhibits a signal drift of no more than 1% of signal/hour.

106. The glucose sensor of claim 105, wherein signal drift is assayed at least four days into a testing regimen.

107. The glucose sensor of claim 105, wherein signal drift is assayed against a concentration of glucose of about 400 mg/dL.

108. The glucose sensor of claim 105, wherein signal drift is assayed against a concentration of glucose of at least 100 mg/dL.

109. The glucose sensor of claim 105, wherein the sensor comprises an insulating layer having a hole distal from an edge of the insulating layer.

110. The glucose sensor of claim 109, wherein the sensor comprises a sensor membrane covalently bound to the insulating layer.

111. The glucose sensor of claim 109, wherein the sensor comprises a sensor membrane covalently bound to an exterior layer.

112. A method of extending sensor performance, comprising affixing a sensor membrane to a sensor core, and binding an exterior coating to the sensor membrane, so as to prevent at least some of the sensor membrane from separating from the sensor core upon introducing a liquid to a sensor.

113. The method of claim 112, wherein the binding is noncovalent binding.

114. The method of claim 112, wherein the binding is covalent binding.

115. The method of claim 112, wherein the sensor exhibits a signal drift of no more than 1% of signal/hour.

116. The method of claim 115, wherein signal drift is assayed at least four days into a testing regimen.

117. The method of claim 115, wherein signal drift is assayed against a concentration of ana analyte of about 400 mg/dL.

118. The method of claim 115, wherein signal drift is assayed against a concentration of ana analyte of at least 100 mg/dL.

119. A method of detecting an analyte in a sample, comprising creating a voltage bias in the sample, and measuring a current passing to a conductive base through at least one opening in an insulating layer, wherein the at least one opening does not share a border with an exterior edge of the insulating layer.

120. The method of claim 119, wherein the sample is a body fluid.

121. The method of claim 119, wherein the detecting is performed on a fluid internal to an individual.

122. The method of claim 119, wherein the voltage bias is no more than 1 volt.

123. The method of claim 119, wherein the voltage bias is no more than 0.6 volt.

124. The method of claim 119, wherein the voltage bias is no more than 0.3 volt.

125. The method of claim 119, wherein the current arises from hydrogen peroxide contacting the conductive base.

126. The method of claim 119, wherein the current arises from oxidation of the analyte.

127. The method of claim 126, wherein the current arises from an enzymatic reaction on the analyte.

128. The method of claim 127, wherein the enzymatic reaction comprises action of an oxidase.

129. The method of claim 127, wherein the enzymatic reaction comprises action of glucose oxidase.

130. The method of claim 127, wherein the enzymatic reaction comprises action of lactate oxidase.

131. The method of claim 126, wherein the analyte comprises glucose.

132. The method of claim 126, wherein the analyte comprises lactate.

133. The method of claim 119, comprising subjecting the analyte to a reaction to generate the current.

134. The method of claim 133, wherein the reaction comprises oxidation.

135. The method of claim 134, wherein the oxidation comprises enzymatic oxidation.

136. The method of claim 135, wherein the oxidation yields a reactive oxygen species.

137. The method of claim 135, wherein the oxidation yields hydrogen peroxide.

138. The method of claim 137, wherein the oxidation yields the hydrogen peroxide in proportion to the amount of the analyte.

139. The method of claim 133, wherein the analyte comprises glucose.

140. The method of claim 133, wherein the analyte comprises lactate.

141. The method of claim 119, wherein the current has an amplitude that is proportional to concentration of the analyte.

142. The method of claim 119, wherein the current has an amplitude that is proportional to amount of the analyte.

143. The method of claim 141 or claim 142, wherein the analyte comprises glucose.

144. The method of claim 141 or claim 142, wherein the analyte comprises lactate.

145. The method of claim 119, wherein the conductive base directs the current to a detector.

146. The method of claim 119, wherein the conductive base comprises a channel that directs the current to a detector.

147. The method of claim 119, comprising limiting diffusion of the analyte to the opening.

148. The method of claim 147, wherein limiting diffusion comprises covering the opening with an external layer.

149. The method of claim 148, wherein the exterior layer is permeable to the analyte.

150. The method of claim 148, wherein the exterior layer is semipermeable to the analyte.

151. The method of claim 119, wherein the insulating layer precludes current from passing to the conductive base other than through the at least one opening.

152. The method of any one of claims 119 to 151, wherein the detecting is performed for at least 2 days.

153. The method of any one of claims 119 to 151, wherein the detecting is performed for at least 4 days.

154. The method of claim 119, wherein the detecting exhibits a signal drift of no more than 1% of signal/hour.

155. The method of claim 119, wherein the detecting exhibits a signal drift of no more than 1% of signal/hour after four days of monitoring.

156. The method of any one of claims 119 to 155, wherein the measuring a current passing to a conductive base through at least one opening in an insulating layer, wherein the at least one opening does not share a border with an exterior edge of the insulating layer is performed by a sensor or any one of claims 1 to 59.
